SEQUENCE LISTING (12 sequences)

SEQ ID NO: 1 - DNA, 115 bp, synthetic construct
  source 1..115: mol_type = other DNA
  misc_feature 1..115: note = aptamer for CSF biomarkers
cgacgcgctgtcccgggcctcggcctggcacaatgcgtgcggcctttaagactcaggttggagcacgagtgtgaaggtcgtatcgctccatgctaggtgccgtaagtgatctccc

SEQ ID NO: 2 - DNA, 97 bp, synthetic construct
  source 1..97: mol_type = other DNA
  misc_feature 1..97: note = aptamer for CSF biomarkers
cgacgcgctgtcccgcacagccgtgagaagactcgacgttaatgagtagatccccgtcaccaacacgcaaaaggttaggtgccgtaagtgatctccc

SEQ ID NO: 3 - DNA, 97 bp, synthetic construct
  source 1..97: mol_type = other DNA
  misc_feature 1..97: note = aptamer for CSF biomarkers
cgacgcgctgtcccgcccacagaaccacagcggtaaatattttaaacttgacaagcagtaacaaagggagacaccaaggtgccgtaagtgatctccc

SEQ ID NO: 4 - DNA, 97 bp, synthetic construct
  source 1..97: mol_type = other DNA
  misc_feature 1..97: note = aptamer for CSF biomarkers
cgacgcgctgtcccgccacgacacgcaaaaataaaaagggaaacgaactaagcagaacagtgaacgatgacatgataggtgccgtaagtgatctccc

SEQ ID NO: 5 - DNA, 97 bp, synthetic construct
  source 1..97: mol_type = other DNA
  misc_feature 1..97: note = aptamer for CSF biomarkers
cgacgcgctgtcccgccacagcgtgggacgacactcggtccagcatacaatcaaggtgcaaacctggtacatgtcggggtgccgtaagtgatctccc

SEQ ID NO: 6 - DNA, 97 bp, synthetic construct
  source 1..97: mol_type = other DNA
  misc_feature 1..97: note = aptamer for CSF biomarkers
cgacgcgctgtcccgcacggacagacgggaattaactcaacgcccaaggagaaggatcactatcgagttaagaggatggtgccgtaagtgatctccc

SEQ ID NO: 7 - DNA, 38 bp, synthetic construct
  source 1..38: mol_type = other DNA
  misc_feature 1..38: note = DNA library
actcgacgcgctgtcccnggtgccgtaagtgatctccc

SEQ ID NO: 8 - DNA, 10 bp, synthetic construct
  source 1..10: mol_type = other DNA
  modified_base <1: mod_base = OTHER; note = 5' Biotin modification
  modified_base <1: mod_base = OTHER; note = Int Spacer 18 modification
  modified_base <1: mod_base = OTHER; note = Int Spacer 18 Modification
  modified_base <1: mod_base = OTHER; note = Int Spacer 18 Modification
  misc_feature <1..10: note = Capture Probe
cgcgtcgagt

SEQ ID NO: 9 - DNA, 17 bp, synthetic construct
  source 1..17: mol_type = other DNA
  modified_base <1: mod_base = OTHER; note = Methylene Blue Modification
  misc_feature <1..17: note = Methylene Blue Primer
actcgacgcgctgtccc

SEQ ID NO: 10 - DNA, 17 bp, synthetic construct
  source 1..17: mol_type = other DNA
  modified_base <1: mod_base = OTHER; note = Cy3 Modification
  misc_feature <1..17: note = Cy3 Primer
actcgacgcgctgtccc

SEQ ID NO: 11 - DNA, 20 bp, synthetic construct
  source 1..20: mol_type = other DNA
  misc_feature 1..20: note = Forward Primer
gggagatcacttacggcacc

SEQ ID NO: 12 - DNA, 15 bp, synthetic construct
  source 1..15: mol_type = other DNA
  misc_feature 1..15: note = Reverse Primer
cgacgcgctgtcccg

(19) United States
(12) Patent Application Publication
(10) Pub. No.: US 2024/0255462 A1
(43) Pub. Date: Aug. 1, 2024

(54) ELECTROCHEMICAL, APTAMER-BASED POINT-OF-CARE CEREBROSPINAL FLUID DETECTION

(71) Applicant: THE REGENTS OF THE UNIVERSITY OF CALIFORNIA, Oakland, CA (US)

(72) Inventors: Edward C. Kuan, Orange, CA (US); Michelle Khine, Irvine, CA (US); Andrej Luptak, Irvine, CA (US); Arash Abiri, Irvine, CA (US); Julia A. Zakashansky, Irvine, CA (US); Xinlei Chen, Irvine, CA (US)

(21) Appl. No.: 18/601,656

(22) Filed: Mar. 11, 2024

Related U.S. Application Data
(63) Continuation-in-part of application No. 18/492,000, filed on Oct. 23, 2023.
(60) Provisional application No. 63/380,554, filed on Oct. 21, 2022.

(51) Int. Cl. G01N 27/327; C12N 15/115; G16C 20/20

(52) U.S. Cl. CPC G01N 27/3277; C12N 15/115; G16C 20/20; C12N 2310/16

(57) ABSTRACT

A portable device for detection of cerebrospinal fluid (CSF) in a sample. The device may comprise a substrate and a sensor disposed on the substrate, the sensor comprising a sensing area. The sensor further comprises an aptamer solution comprising an aptamer sequence functionalized by a redox agent to be sensitive to the CSF biomarker and functionalized to attach to the sensor. The sensor may be configured to be sensitive to a CSF biomarker. The device may further comprise an electrochemical detection component operatively coupled to the sensor. When a sample containing an amount of the CSF biomarker is disposed on the sensing area of the sensor, the electrode may generate a detection signal to be transmitted to the electrochemical detection component. The electrochemical detection component may transmit the detection signal to the computing device.

### CROSS-REFERENCES TO RELATED APPLICATIONS

This application is a continuation-in-part and claims priority to U.S. Non-Provisional patent application Ser. No. 18/492,000 filed on Oct. 23, 2023, which is a non-provisional and claims priority to U.S. Provisional Patent Application No. 63/380,554 filed on Oct. 21, 2022, the specification of which is incorporated herein in its entirety by reference.

### REFERENCE TO A SEQUENCE LISTING

The contents of the electronic sequence listing (UCI_22_26_CIP.xml; Size: 20,029 bytes; and Date of Creation: 3-11-2024) is herein incorporated by reference in its entirety.

### FIELD OF THE INVENTION

The present invention is directed to rapid, portable devices for detecting cerebrospinal fluid in a sample through the use of aptamer solutions and wrinkled-film electrodes.

### BACKGROUND OF THE INVENTION

Cerebrospinal fluid (CSF) is the clear, water-like liquid that normally bathes the brain and spinal cord, and provides buoyancy and nutrient transportation to these vital structures. CSF leak, which heralds a breach between the cranial or spinal cavity and the outside world, is increasingly recognized as an important class of disorders that can be life-threatening and cause a tremendous negative impact on quality of life. Classically, CSF leaks tend to occur in the context of craniofacial trauma; surgical complication; or congenital encephaloceles (outpouching of brain and/or dural tissue into the nose or ear) (FIGS. 2A-2D). More recently, spontaneous violations of the skull base due to increased intracranial pressure (ICP), which is linked to elevated body weight, have been identified as another etiology for the development of CSF leaks. Additionally, with increasing utilization of minimally invasive (i.e., endoscopic) techniques to access various skull base pathologies, surgically created defects, which frequently lead to CSF leak, require reconstruction to seal off the intracranial space from the sinonasal cavity to prevent intracranial infection. There is mounting evidence to suggest that the incidence of CSF leaks is rising, partly due to the obesity epidemic and partly due to increased endoscopic procedures, and there is growing recognition of the need to better diagnose and manage CSF leaks.

Despite advances in skull base reconstruction, CSF leak remains a concerning postoperative complication in skull base surgery. Delayed recognition of active CSF leak can lead to severe and potentially life-threatening intracranial infections, particularly meningitis. Moreover, CSF leaks are associated with a tremendous socioeconomic burden, with additional diagnostic and therapeutic costs of $15,000-$66,000 per patient. Therefore, prompt and accurate diagnosis of CSF leaks is critical to delivering high-quality patient care, especially in view of the seemingly rising incidence of such pathologies.

The current gold standard technique to diagnose suspected CSF leaks involves beta-2 transferrin (β2TF) immunofixation. Transferrin, a glycoprotein essential for iron homeostasis, is present in multiple forms within the human body. In the brain, its neuraminidase-eliminated terminal sialic acid residues on the glycan chain result in asialo-transferrin, also known as β2TF, which constitutes approximately one-third of total transferrin in the CSF. Among various biochemical assays used for detecting CSF, β2TF is regarded as the most reliable and specific biomarker. Its absence in physiologic nasal/otic secretions or blood makes it an ideal biomarker, hence its use in various techniques such as gel electrophoresis, immunofixation, or isoelectric focusing for detecting CSF with as high as 100% sensitivity and 94% specificity, though false negative results are certainly possible. Current state-of-the-art technology allows for accurate β2TF detection via electrophoresis; however, this technique's accessibility and practicality have been largely hindered by its 3-7 day latency time and need for sample purity, adequate sample quantity (at least 1 mL, which may take time to acquire when CSF leaks are low-flow or drop-like), and skilled professionals at a specialized laboratory.

For this reason, other noninvasive quantitative methods that involve measuring glucose, total protein, or prealbumin concentrations in nasal drainage have since been developed to serve as alternatives for diagnosing CSF leaks. However, these tests have been found to be nonspecific and are not recommended for guiding medical management. Recently, Bradbury et al. published their experience developing a point-of-care (POC) CSF detection device based on quantifying CSF-specific protein concentration. However, this technique relied on extensive sample pre-processing by a skilled technician, signaling via liquid color change, had a relatively high limit of detection (˜1 mg/L) and demonstrated ambiguous results in samples contaminated by blood. Additionally, the aforementioned device utilized beta trace protein (BTP), which, though commonly used in Europe, is also present in the serum, heart, and testes, and thus may be prone to false positive results. Other attempts at developing POC technologies have utilized novel, though nonstandard and unvalidated, molecular targets, and/or have not been tested in the setting of contaminated samples. Importantly, there has been no systematic evaluation of how the presence of common contaminants (e.g., blood, mucus, saliva), which are largely unavoidable when sampling CSF in actual clinical settings, impact the accuracy of detection. Thus, there exists a present need for an alternative portable technology that utilizes an electrochemical-aptamer-based (E-AB) approach to quickly, specifically, and accurately detect CSF using a small sample volume without the need to pre-treat samples, regardless of sample contamination with blood or secretions, with pilot clinical validation in the real-world setting compared against the standard of care.

### BRIEF SUMMARY OF THE INVENTION

It is an objective of the present invention to provide systems and methods that allow for portable devices for detecting cerebrospinal fluid in a sample through the use of aptamer solutions and wrinkled-film electrodes, as specified in the independent claims. Embodiments of the invention are given in the dependent claims. Embodiments of the present invention can be freely combined with each other if they are not mutually exclusive.

The present invention is directed to a “lab-on-a-chip” point-of-care (POC) device based on the proprietary highly sensitive and specific electrochemical-aptamer-based (E-AB) electrodes. The present invention utilizes custom-designed aptamers specific to CSF biomarkers in combination with a miniaturized electrochemical chip design to serve as a POC device for detecting cerebrospinal fluid (CSF). The non-invasive device may enable rapid (within minutes) and accurate CSF detection with low sampling volume, despite possible contamination of clinical specimens with other bodily fluids (e.g., blood mucus). The utility of this device will be analogous to the highly accessible and portable glucometer, which provides fast and accurate results with low sampling volumes.

This project aims to fill the current gap of prompt and accurate POC CSF leak detection in the context of low sample volumes and sample contamination by other bodily fluids. Despite this critical need, there currently exists no FDA-approved POC device for this purpose, and efforts at the forefront of CSF detection are still limited to purified or heavily pretreated samples, with no accurate detection on untreated or contaminated human CSF samples. The innovation at the interdisciplinary interface of otolaryngology/neurosurgery, biomedical engineering, and pharmaceutical sciences implements aptamer selection to develop a scalable, point-of-care technology to rapidly and specifically detect the presence of CSF-specific biomarkers for clinical applications. The present invention utilizes a method for the direct selection of conformation-switching DNA aptamers, which not only bind target molecules (e.g., CSF-specific biomarkers), but the binding event is also directly coupled to a readout such that a diagnostic molecular tool can be directly built using the aptamer. In addition, this innovation will heavily benefit from the novel and low-cost shrink-induced wrinkled gold plate design, which, through its micro- and nanostructured surface topography, increases the surface area for the working electrodes and thereby provides a significantly greater electrochemical signal per macroscopic unit area for achieving higher sensitivity and specificity in CSF-specific biomarker detection.

The present invention features a portable device for the detection of cerebrospinal fluid (CSF) in a sample. In some embodiments, the device may comprise a substrate and a sensor disposed on the substrate, the sensor comprising a sensing area. The sensor may be incubated with an aptamer solution. The sensor may be configured to be sensitive to a CSF biomarker. The device may further comprise a potentiostat operatively coupled to the sensor. The potentiostat may be configured to connect to a computing device. When a sample containing an amount of the CSF biomarker is disposed in the sensing area of the sensor, the electrode may generate a detection signal to be transmitted to the potentiostat. The potentiostat may transmit the detection signal to the computing device.

The present invention features a method for detecting cerebrospinal fluid (CSF) in a sample. In some embodiments, the method may comprise providing a device comprising a substrate and a sensor disposed on the substrate, the sensor comprising a sensing area. The sensor may be incubated with an aptamer solution, and the sensor may be configured to be sensitive to a CSF biomarker. The device may further comprise a potentiostat operatively coupled to the sensor. The potentiostat may be configured to connect to a computing device. The method may further comprise applying a sample to the sensing area of the sensor, generating, by the sensor, a detection signal if the sample contains an amount of the CSF biomarker, transmitting the detection signal to the potentiostat, and transmitting, by the potentiostat, the detection signal to the computing device.

The present invention is additionally directed to a single-stranded deoxyribonucleic acid (ssDNA) aptamer capable of selectively binding to CSF-specific biomarkers, for the development of an aptamer-based POC CSF detection device.

One of the unique and inventive technical features of the present invention is the implementation of wrinkled gold electrodes incubated with an aptamer solution such that the electrodes are sensitive to CSF biomarkers. Without wishing to limit the invention to any theory or mechanism, it is believed that the technical feature of the present invention advantageously provides for fast, efficient, accurate, and portable detection of small amounts of CSF in a sample. None of the presently known prior references or work has the unique inventive technical feature of the present invention.

Prior works, such as U.S. Pat. No. 11,209,427, teach devices and methods for determining the presence (or absence) of a cerebrospinal fluid (CSF) leak by utilizing a semi-quantitative, barcode-style lateral-flow immunoassay (LFA) that quantifies the level of beta-trace protein (BTP) in a sample. However, this prior work teaches an antibody-based method for modifying an electrode to detect CSF, whereas the present invention implements an aptamer-based method. Because of this, the prior work requires multiple steps for lateral flow assay whereas the present invention only requires a sample to be plugged into the device to generate a detection signal.

Another one of the unique and inventive technical features of the present invention is the implementation of nucleotide sequences that yield aptamers capable of binding to molecules within CSF more favorably than molecules within other common physiological fluids. Without wishing to limit the invention to any theory or mechanism, it is believed that the technical feature of the present invention advantageously provides for more accurate detection of small amounts of CSF in a sample without binding to other fluids on accident. None of the presently known prior references or work has the unique inventive technical feature of the present invention.

Any feature or combination of features described herein are included within the scope of the present invention provided that the features included in any such combination are not mutually inconsistent as will be apparent from the context, this specification, and the knowledge of one of ordinary skill in the art. Additional advantages and aspects of the present invention are apparent in the following detailed description and claims.

### BRIEF DESCRIPTION OF THE SEVERAL VIEWS OF THE DRAWING(S)

The patent application or application file contains at least one drawing executed in color. Copies of this patent or patent application publication with color drawing(s) will be provided by the Office upon request and payment of the necessary fee.

The features and advantages of the present invention will become apparent from a consideration of the following detailed description presented in connection with the accompanying drawings in which:

FIG. 1A shows a schematic for a handheld potentiostat that interfaces with the sensor for data acquisition.

FIG. 1B shows a flow chart for a method for detecting cerebrospinal fluid (CSF) in a sample.

FIG. 1C shows a process flow to create shrink-induced wrinkled E-AB sensors. First, a thin layer of gold is sputtered onto polystyrene plastic, which is shrunk to create wrinkles (SEM inset shows representative wrinkle morphology). The wrinkled surface is incubated with aptamers conjugated with methylene blue (MB), and then incubated with 6-mercaptohexanol (MCH) as the blocking molecule. After functionalization, the wrinkled surface was exposed to a CSF biomarker. The graph illustrates the change in current due to the change in electron transfer for CSF biomarker-bound MB on wrinkled electrodes upon the addition of the CSF biomarker.

FIG. 1D shows a schematic of the handheld potentiostat of the present invention.

FIG. 2A shows a patient with an active CSF leak dripping from the nose (note the thin, clear water-like consistency of the fluid that can mix with nasal secretions or blood in the nose).

FIG. 2B shows sagittal computed tomography (CT) imaging demonstrating a highly comminuted posterior table of frontal sinus/skull base fracture (*) from craniofacial trauma leading to pneumocephalus and CSF leak.

FIG. 2C shows a coronal CT scan of a patient with left frontoethmoidal skull base defect (*) and herniation of meningeal tissue into the sinus cavity due to intracranial hypertension, with active CSF leak.

FIG. 2D shows an endoscopic view of meningoencephalocele within the right cribriform plate (*) flanked between the nasal septum (S) and right middle turbinate (MT) as a result of intracranial hypertension.

FIG. 3 shows photos of unshrunk (left) and shrunk (right) gold electrodes.

FIG. 4 shows the In vitro selection of structure-switching aptamers. A random-sequence DNA library modified with methylene blue (MB) at the 5′ terminus was immobilized on magnetic beads via biotinylated capture oligo. Plasma was used to wash off ssDNA with low specificity for CSF biomarkers (i.e., binders of non-CSF proteins). Upon adding CSF, the CSF-specific ssDNA underwent a biomarker-dependent conformational change, binding to the target and “switching off” from the beads. This ssDNA unbound from the beads was isolated and amplified to become the new library for the next round of selection. After 7 rounds of selection, the enriched library was sequenced. The most abundant ssDNA sequence was selected for further characterization.

FIG. 5 shows the qPCR experimental workflow. The ssDNA was bound to streptavidin beads and excess unbounded ssDNA was washed with PBS twice. The remaining bounded ssDNA was washed with plasma twice before separating into three tubes. Each tube was washed with a CSF sample from a different patient. DNA samples were extracted from all washes (pink) and quantified by qPCR.

FIG. 6 shows a flow chart of fabricating a wrinkled film electrode sensitive to a CSF biomarker. Upon heating, gold-sputtered polystyrene shrinks and creates a wrinkled gold surface (SEM insert shows wrinkle morphology). Electrodes are defined by laser ablation of the gold film to remove the gold not defined by the shadow mask. Then, the Ag/AgCI electrode is screen-printed on the polystyrene surface. The MB-labeled aptamer is attached to gold via thiolation. After functionalization, the wrinkled surface is exposed to the target. The target-induced conformational change of the aptamer changes the electron transfer of the MB, resulting in a change in current.

FIG. 7 shows a proposed cartridge system that integrates with the electrode chip and fits into a potentiostat, the system comprising a reagent reservoir, a sample reservoir, microfluidics, an electrode chip that slides into the cartridge, and a potentiostat.

FIG. 8 shows a graph showing the aptamers of the present invention binding to different targets, one <100 kDa in size and the other >100 kDa in size.

FIG. 9A shows the evolution of reads per million (RPM) counts of the top 10 most abundant clusters from selection rounds 7-14. The top 10 clusters were based on sequencing data from round 14.

FIG. 9B shows the RPM ratio of CSF wash to serum wash on the top 10 clusters from cycle 14. The black dotted line indicates an RPM ratio of 1. A ratio greater than 1 indicated that the cluster sequence was more abundant in the CSF wash than in the serum wash.

FIG. 10A shows the qPCR experimental protocol. The ssDNA was bound to streptavidin beads and excess unbounded ssDNA was washed with DPBS twice. The remaining bounded ssDNA was washed with serum, followed by DPBS and then CSF. DNA samples were extracted from all washes and quantified by qPCR.

FIG. 10B shows quantified ssDNA concentrations for washes involving C1. Concentrations of C1 were 10× higher in CSF compared to serum washes.

FIG. 10C shows quantified ssDNA concentrations for washes involving C2. Concentrations of C2 were 586× higher in CSF compared to serum washes.

FIG. 10D shows quantified ssDNA concentrations for washes involving C3. Concentrations of C3 were 82× higher in CSF compared to serum washes.

FIG. 11A shows secondary structure predictions for the C2 sequence, demonstrating possible unpaired regions (orange) and sites of hairpin (blue) or interior loops (yellow).

FIG. 11B shows quantified ssDNA concentrations of C2 following washing with size-exclusion (<100 kDa or >100 kDa) or whole CSF. A higher concentration of C2 was detected in <100 kDa CSF than >100 kDa CSF.

FIG. 11C shows secondary structure predictions for the C3 sequence, demonstrating possible unpaired regions (orange) and sites of hairpin (blue) or interior loops (yellow).

FIG. 11D shows quantified ssDNA concentrations of C3 following washing with size-exclusion (<100 kDa or >100 kDa) or whole CSF. A higher concentration of C3 was detected in >100 kDa CSF than in <100 kDa CSF.

FIG. 12 shows a graph of fluorescence anisotropy of Cy3-labeled C2 and C3 ssDNA after 2-minute incubation with varying CSF concentrations. A random cy3-labeled 98-nt oligonucleotide uninvolved in the selection protocol was used as a negative control (NC). With higher CSF concentrations, C2 and C3 exhibited increases in anisotropy while the NC oligonucleotide did not.

### DETAILED DESCRIPTION OF THE INVENTION

Following is a list of elements corresponding to a particular element referred to herein:
100 device
110 substrate
120 sensor
130 electrochemical detection component
140 computing device

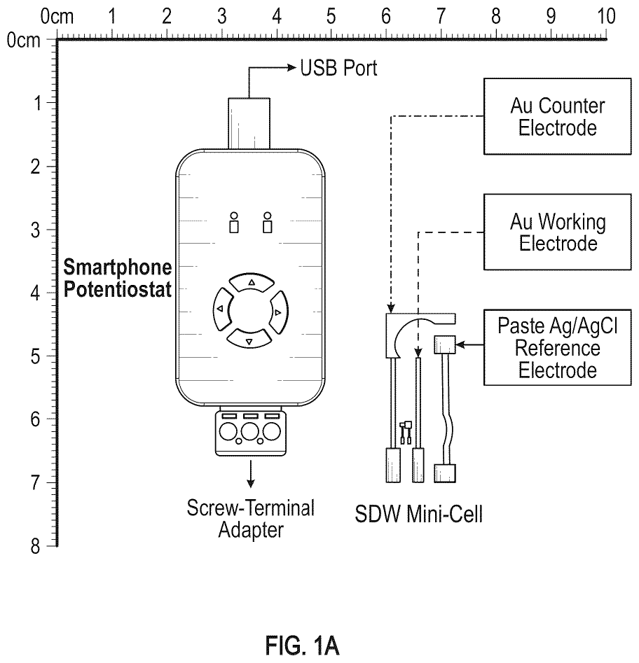

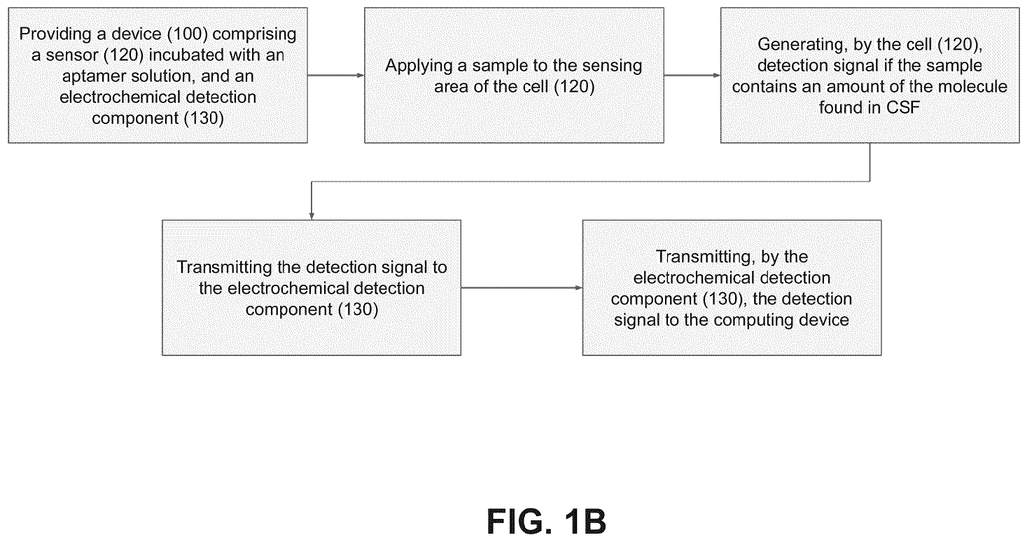

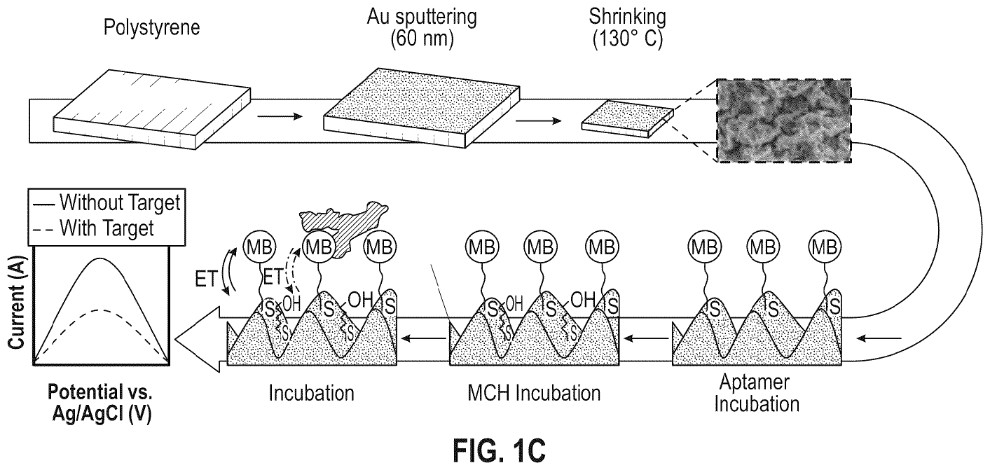

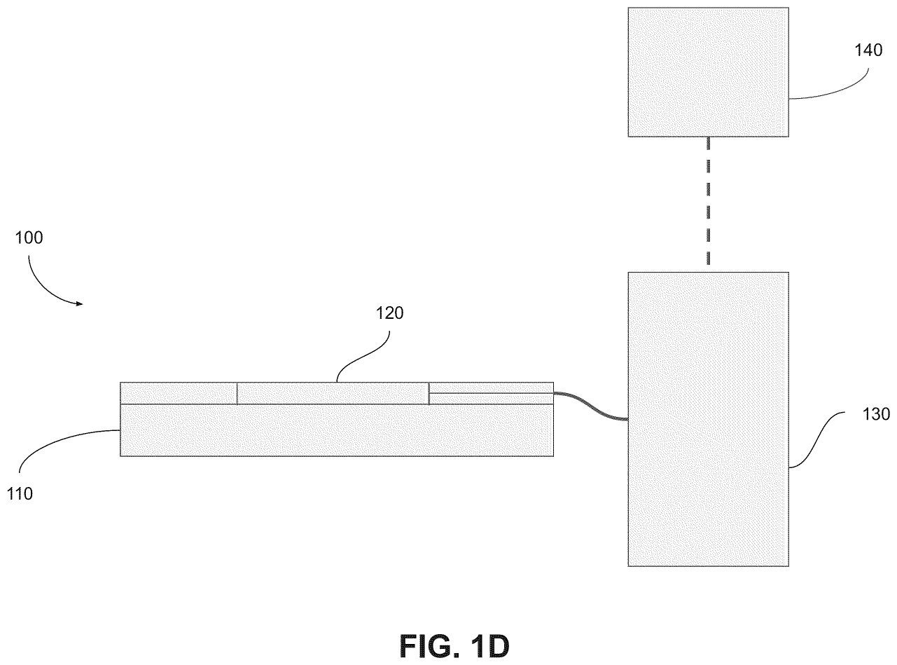

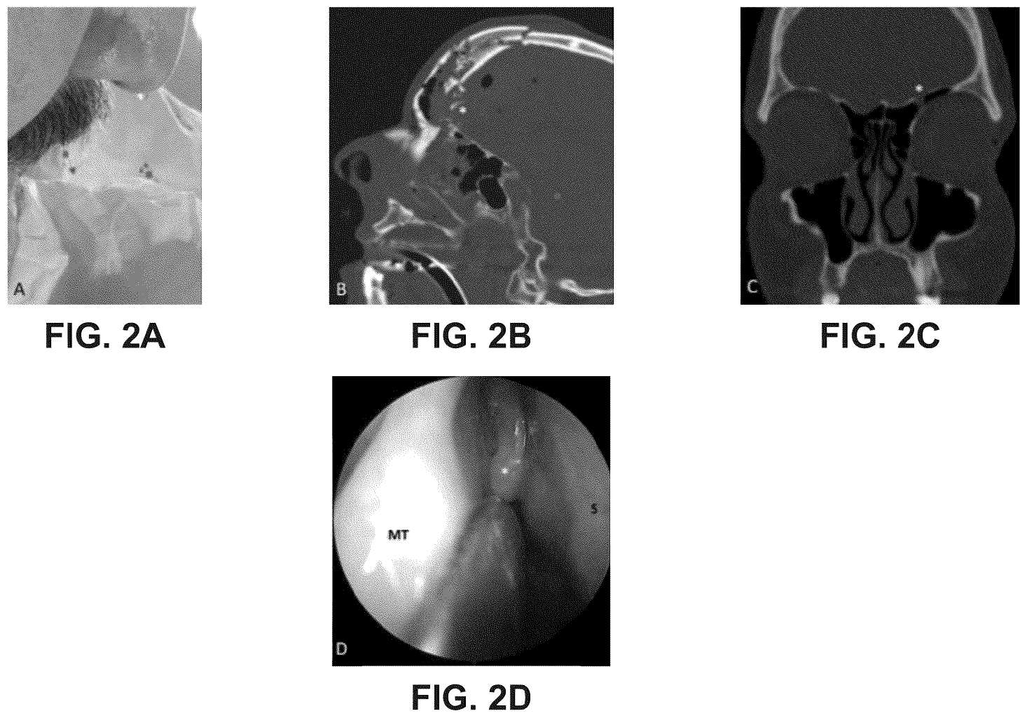

Referring now to FIGS. 1A and 1D, the present invention features a portable device (100) for detection of cerebrospinal fluid (CSF) in a sample. In some embodiments, the device (100) may comprise a substrate (110) and a miniaturized electrochemical sensor (120) disposed on the substrate (110), the sensor (120) comprising a sensing area. The sensor (120) may comprise an aptamer solution comprising an aptamer sequence comprising a first end and a second end. The first end is functionalized by a redox agent to be sensitive to one or more molecules found in CSF, and the second end is functionalized to attach to a surface of the sensor (120). The sensor (120) may be configured to be sensitive to the one or more molecules found in CSF. The device (100) may further comprise an electrochemical detection component (130) operatively coupled to the sensor (120). The electrochemical detection component (130) may be configured to connect to a computing device (140). When a sample containing an amount of the one or more molecules found in CSF is disposed on the sensing area of the sensor (120), the electrode may generate a detection signal to be transmitted to the electrochemical detection component (130). The electrochemical detection component (130) may transmit the detection signal to the computing device (140).

In some embodiments, the sensor (120) may comprise a miniaturized reference electrode, a miniaturized working electrode, and a miniaturized counter electrode. In some embodiments, the miniaturized electrodes may comprise a wrinkled reference electrode, a wrinkled working electrode, and a wrinkled counter electrode. The wrinkled reference electrode, the wrinkled working electrode, and the wrinkled counter electrode may each comprise a thin shrunken layer of gold sputtered onto polystyrene plastic. In other embodiments, the miniaturized electrodes may comprise nanoparticle electrodes. In some embodiments, the aptamer solution is modified by a redox agent. The redox agent may comprise methylene blue, ferrocene, or a combination thereof. In some embodiments, the computing device (140) may comprise a personal computer, a portable computing device, or a cloud computing device. In some embodiments, the electrode may be capable of sensing 1 ag/mL or more of the one or more molecules found in CSF. The aptamer may be one or a combination of SEQ ID NO: 1-6. In some embodiments, the electrochemical detection component comprises a potentiostat, a multimeter, or a combination thereof.

Referring now to FIG. 1B, the present invention features a method for detecting cerebrospinal fluid (CSF) in a sample. In some embodiments, the method may comprise providing a device (100) comprising a substrate (110) and a miniaturized electrochemical sensor (120) disposed on the substrate (110), the sensor (120) comprising a sensing area. The sensor (120) may comprise an aptamer solution comprising an aptamer sequence comprising a first end and a second end. The first end is functionalized by a redox agent to be sensitive to one or more molecules found in CSF, and the second end is functionalized to attach to a surface of the sensor (120), and the sensor (120) may be configured to be sensitive to the one or more molecules found in CSF. The device (100) may further comprise an electrochemical detection component (130) operatively coupled to the sensor (120). The electrochemical detection component (130) may be configured to connect to a computing device (140). The method may further comprise applying a sample to the sensing area of the sensor (120), generating, by the sensor (120), a detection signal if the sample contains an amount of the one or more molecules found in CSF, transmitting the detection signal to the electrochemical detection component (130), and transmitting, by the electrochemical detection component (130), the detection signal to the computing device (140).

In some embodiments, the sensor (120) may comprise a miniaturized reference electrode, a miniaturized working electrode, and a miniaturized counter electrode. In some embodiments, the miniaturized electrodes may comprise a wrinkled reference electrode, a wrinkled working electrode, and a wrinkled counter electrode. The wrinkled reference electrode, the wrinkled working electrode, and the wrinkled counter electrode may each comprise a thin shrunken layer of gold sputtered onto polystyrene plastic. In other embodiments, the miniaturized electrodes may comprise nanoparticle electrodes. In some embodiments, the aptamer solution is modified by a redox agent. The redox agent may comprise methylene blue, ferrocene, or a combination thereof. In some embodiments, the computing device (140) may comprise a personal computer, a portable computing device, or a cloud computing device. In some embodiments, the electrode may be capable of sensing 1 ag/mL or more of the one or more molecules found in CSF. The aptamer may be one or a combination of SEQ ID NO: 1-6. In some embodiments, the electrochemical detection component comprises a potentiostat, a multimeter, or a combination thereof.

Referring now to FIG. 1C, the present invention features a method for fabricating a miniaturized electrochemical sensor (120) sensitive to one or more molecules found in CSF. In some embodiments, the method may comprise providing a substrate (110) comprising a thin layer of gold, shrinking the substrate (110), incubating the substrate (110) with an aptamer solution, and exposing the substrate (110) to the one or more molecules found in CSF such that the substrate (110) is capable of generating a signal in response to being exposed to the one or more molecules found in CSF. In some embodiments, the aptamer solution is modified by a redox agent. The redox agent may comprise methylene blue, ferrocene, or a combination thereof. In some embodiments, the aptamer solution comprises aptamers having a sequence according to one of SEQ ID NO: 1-6. In some embodiments, the electrode may be capable of sensing 1 ag/mL or more of the one or more molecules found in CSF. In some embodiments, the substrate (110) may comprise polystyrene plastic.

The present invention features an aptamer sequence comprising a first end and a second end. The first end may be functionalized by a redox agent to be sensitive to one or more molecules found in cerebrospinal fluid (CSF), and the second end may be functionalized to attach to a surface of an electrode. The redox agent may comprise methylene blue, ferrocene, any chemical capable of causing the aptamer to change electron transfer in response to the one or more molecules found in CSF, or a combination thereof. The second end may be functionalized by thiolization, biotinylation, or a combination thereof. The present invention features a composition comprising one or more aptamers according to SEQ ID NO: 1-6.

In some embodiments, the substrate may have a thickness of 1.4 to 1.6 mm. In some embodiments, the sensor may have a thickness of 120 to 140 nm. In some embodiments, the sensor may communicate with the electrochemical detection component by a wired connection, a wireless connection, or a combination thereof. In some embodiments, the electrochemical detection component may communicate with the computing device by a wired connection, a wireless connection, or a combination thereof. In some embodiments, the sensing area of the sensor may comprise 1% to 3% of the area of the sensor. In some embodiments, the reference electrode, working electrode, and counter electrode of the sensor may be arranged such that the working electrode is at the center, while the reference electrode and counter electrode are on the sides (see FIG. 1A).

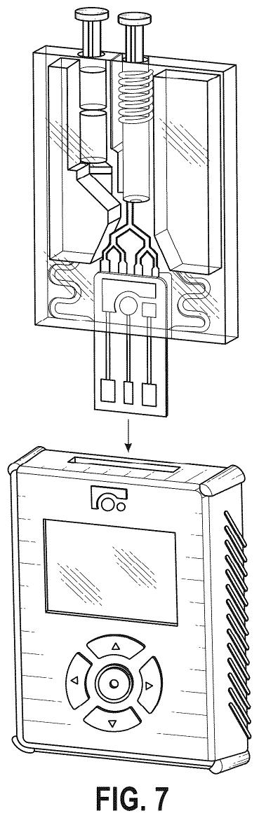

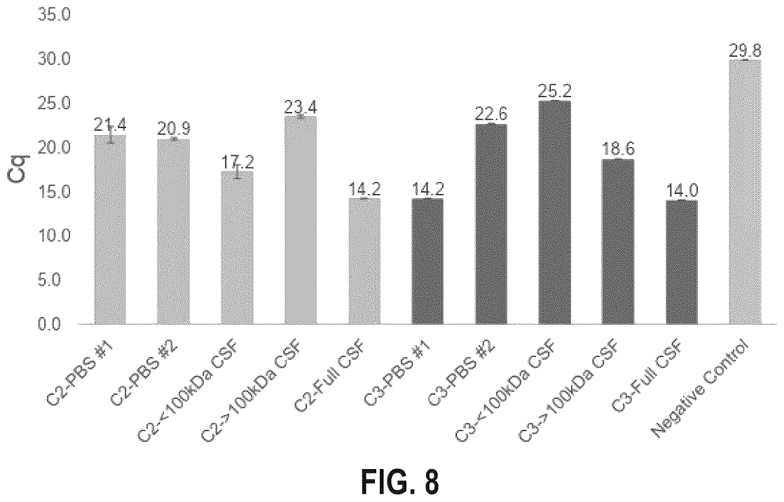

Referring now to FIG. 7, the present invention features a cartridge-based system for collecting the sample and generating an output. In some embodiments, the cartridge-based system may comprise a cartridge component configured to accept the sample (e.g. fluid from the patient). In some embodiments, the cartridge component may comprise or may be removably coupled to a sample reservoir in the system. The system may further comprise a reagent reservoir configured to contain the aptamer of the present invention and apply it to the cartridge. In some embodiments, the aptamer and the sample may be configured to travel from the reagent reservoir and the sample reservoir, respectively, to an electrode chip through one or more microfluidic channels. In some embodiments, the electrode chip may be configured to slide into the cartridge. The system may further comprise a potentiostat, configured to control the electrode chip's potential to control the electrochemical reactions on the electrode chip and measure the binding events of the aptamer to the CSF-specific molecules in the sample.

In some embodiments, the aptamer of the present invention may be configured to one or more target molecules specific to CSF. In some embodiments, the one or more target molecules may comprise molecules having a size of 100 kDa or more. Thus, this aptamer target represents a new CSF-specific molecule targeting aptamer, as prior aptamers do not target molecules having a size greater than or equal to 100 kDa.

In some embodiments, the sensor of the present invention may comprise one or more optical transducers. In some embodiments, the one or more optical transducers may comprise one or more fluorescence-based detectors, suitable if the aptamer can be tagged with a fluorophore or induce a fluorescent response upon binding to the CSF-specific molecule(s). In some embodiments, the one or more optical transducers may comprise one or more surface plasmon resonance transducers for real-time detection of CSF-specific molecule binding at a sensor surface without the need for labeling. In some embodiments, the one or more optical transducers may comprise one or more colorimetric detection transducers for enabling visual detection of CSF-specific molecule binding through the formation of colored complexes.

In some embodiments, the sensor of the present invention may comprise one or more piezoelectric transducers. In some embodiments, the one or more piezoelectric transducers may comprise one or more quartz crystal microbalance transducers for measuring changes in frequency due to binding on the quartz surface.

In some embodiments, the sensor of the present invention may comprise one or more thermal transducers. In some embodiments, the one or more thermal transducers may comprise one or more microcantilever sensors for detecting deflections resulting from differential surface stress upon a binding event of the aptamer to the CSF-specific molecule. In some embodiments, the one or more thermal transducers may comprise one or more calorimetric detection sensors for capturing heat variations during the binding event of the aptamer to the CSF-specific molecule.

In some embodiments, the sensor of the present invention may comprise one or more magnetic transducers. In some embodiments, the one or more magnetic transducers may comprise one or more magnetic bead assays for using aptamers attached to magnetic beads with binding events altering their properties.

In some embodiments, the sensor of the present invention may comprise one or more acoustic wave sensors. In some embodiments, the one or more acoustic wave sensors may be configured to detect variations in wave properties due to mass loading on a substrate as a result of binding events.

In some embodiments, the sensor of the present invention may comprise one or more nanomechanical sensors. In some embodiments, the one or more nanomechanical sensors may comprise nanoscale structures that shift mechanically upon binding events. The one or more nanomechanical sensors may be configured to detect the shifting of the nanoscale structures to measure the presence of CSF-specific molecules.

In some embodiments, the sensor of the present invention may comprise one or more field-effect transistors. In some embodiments, the one or more field-effect transistors may be configured to direct electronic readout from binding events, affecting conductance. This conductance may be measured to determine the presence of CSF-specific molecules.

In vitro selection experiments have been applied for the past three decades to uncover aptamers, ribozymes, and a wide range of regulatory and structural nucleic acids, most notably with diagnostic and therapeutic goals. These nucleic acids are capable of recognizing a wide range of molecules with high affinity and specificity. Aptamers are identified through their highly selective interaction with proteins using an approach known as Systematic Evolution of Ligands by EXponential enrichment (SELEX) or in vitro selection—starting with a target protein with known function, a large and diverse random sequence deoxyribonucleic acid (DNA) library is screened for specific interaction. SELEX screens have identified an FDA-approved aptamer drug to treat macular degeneration. Ten additional aptamers have passed phase I or II clinical trials to treat various diseases, and, due to their versatility, several aptamer-based sensor platforms have been developed.

Aptamers have several favorable properties, including low cost, long-term chemical stability, ease of chemical design, and bulk synthesis, allowing facile incorporation of functional moieties, such as fluorescent dyes and electron donors. The unique stability of DNA makes aptamers available long-term without requiring refrigeration, making them attractive for the development of diagnostic tools. Several in vitro selections have yielded aptamers that specifically bind a single conformation of their target proteins, including the ATP-bound form of the Hsp70 chaperone, 74 Ca2+-bound form of calsenilin, and the β-form of the prion protein. These examples provide a compelling precedent for using in vitro selections to identify conformation-specific aptamers and form a particularly strong intellectual and practical foundation for the development of aptamers that recognize CSF biomarkers, even if they are isoforms of proteins available in other biological fluids (i.e., blood).

Conformation-switching aptamers are a class of aptamers that not only bind their target with tunable affinity and specificity but also change conformation upon binding. In the aptamer sensor system, part of the aptamer domain is initially bound by a short complementary DNA sequence through simple base pairing. When exposed to the target molecule, the aptamers bind the target and undergo a conformational change (“switch”) to release the short sequence. This “switch” can then be coupled to a read-out, such as a fluorophore release, nanoparticle disaggregation with color change, triggering of a hybridization chain reaction, or a change in conductance of an electronic device. The present invention implements in vitro selection of DNA conformation-switching aptamers that recognize biomarkers in CSF. The selected pool of DNAs from CSF samples collected from patients has undergone 7 rounds of selection and, in preliminary experiments, the most enriched sequence exhibits CSF-dependent release from the immobilizing oligonucleotide sequence.

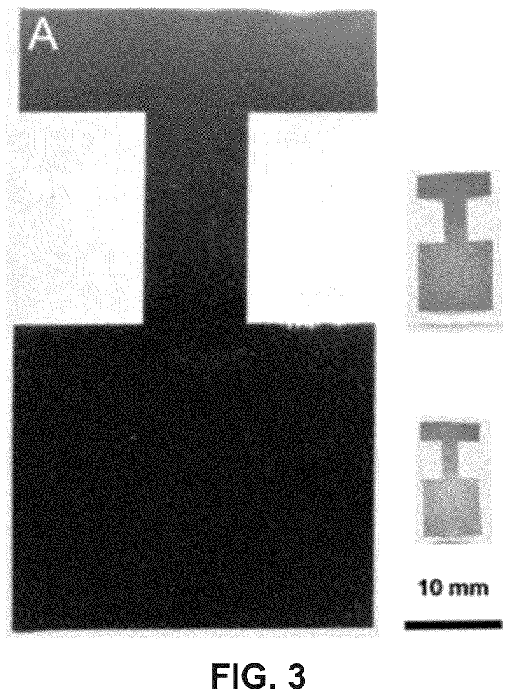

The E-AB model utilizes electrochemical sensing to differentiate molecules based on distinct current densities. As the signal-to-noise ratio is directly correlated with the sensing electrode surface area, retaining a high surface area even in a miniaturized system is critical. The recent implementation of this principle has paved the way for developing novel biosensors capable of detecting various biomarkers such as glucose, uric acid, and lactic acid, with high sensitivity. The present invention has shown the ability to not only dramatically increase electrode surface area-to-volume ratio using a novel shrink-induced miniaturization platform (FIG. 3A), but to also leverage these high surface area sensors to non-enzymatically detect glucose with the lowest limits of detection reported for stretchable sensors. Additionally, the present invention is capable of selectively functionalizing the substrates and applies combinations of electrochemical, chemiluminescent, and plasmonic principles to develop novel biosensors and microfluidics for clinical applications. These shrink-induced high surface area electrodes can be applied to aptamer technology to achieve exceptional signal gains and enhanced signal-to-noise ratios for high-precision SARS-COV-2 spike protein detection in saliva.

The following table describes non-limiting examples of aptamers.

| SEQ | |
|---|---|
| ID | |
| NO: | Sequence |
| 1 | 5′- |
| | cgacgcgctgtcccgggcctcggcctggcacaatgcgtgcggcc |
| | tttaagactcaggttggagcacgagtgtgaaggtcgtatcgctc |
| | catgctaggtgccgtaagtgatctccc-3′ |
| 2 | 5′- |
| | cgacgcgctgtcccgcacagccgtgagaagactcgacgttaatg |
| | agtagatccccgtcaccaacacgcaaaaggttaggtgccgtaag |
| | tgatctccc-3′ |
| 3 | 5′- |
| | cgacgcgctgtcccgcccacagaaccacagcggtaaatatttta |
| | aacttgacaagcagtaacaaagggagacaccaaggtgccgtaag |
| | tgatctccc-3′ |
| 4 | 5′- |
| | cgacgcgctgtcccgccacgacacgcaaaaataaaaagggaaac |
| | gaactaagcagaacagtgaacgatgacatgataggtgccgtaag |
| | tgatctccc-3′ |
| 5 | 5′- |
| | cgacgcgctgtcccgccacagcgtgggacgacactcggtccagc |
| | atacaatcaaggtgcaaacctggtacatgtcggggtgccgtaag |
| | tgatctccc-3′ |
| 6 | 5′- |
| | cgacgcgctgtcccgcacggacagacgggaattaactcaacgcc |
| | caaggagaaggatcactatcgagttaagaggatggtgccgtaag |
| | tgatctccc-3′ |

### EXAMPLE

The following is a non-limiting example of the present invention. It is to be understood that said example is not intended to limit the present invention in any way. Equivalents or substitutes are within the scope of the present invention.

While recent efforts in CSF detection have implemented immunoassays for protein recognition, aptamers have been shown to possess significant advantages over antibodies due to their nucleic acid nature and target-induced structure-switching properties. Furthermore, because they are chemically synthesized through an in vitro process, aptamers not only offer remarkable design flexibility but are inexpensive to produce and are generated to a high degree of purity with little to no batch-to-batch variation. Therefore, synthesizing a DNA aptamer specific to CSF using β2TF as the candidate biomarker will enable the development of a cost-effective E-AB biosensor capable of high-accuracy, high-precision CSF detection.

Identification and characterization of bona fide aptamers can be a slow step during in vitro selection experiments. To facilitate the rapid discovery of aptamers, chemical probing of DNA structures was combined with high-throughput sequencing of selected pools. The approach is analogous to the well-known SHAPE analysis of RNA structure and its application to whole transcriptome analysis but is adapted to DNA using dimethyl sulfate probing. The method reveals: 1) the sequence of each in vitro selected DNA in the pool, 2) the structural features of each sequence (particularly single-stranded loops and bulges, which typically form the binding sites of aptamers), and 3) positions where the structural changes due to target binding in a quantitative manner, revealing the binding site and dissociation coefficient (K_{d}) for each aptamer in the pool. Thus, in a single sequencing run, it is hypothesized that identifying DNAs that bind their targets in solution, measuring their affinities, and pinpointing the segments of these aptamers that facilitate the interactions, vastly increase the throughput of aptamer discovery.

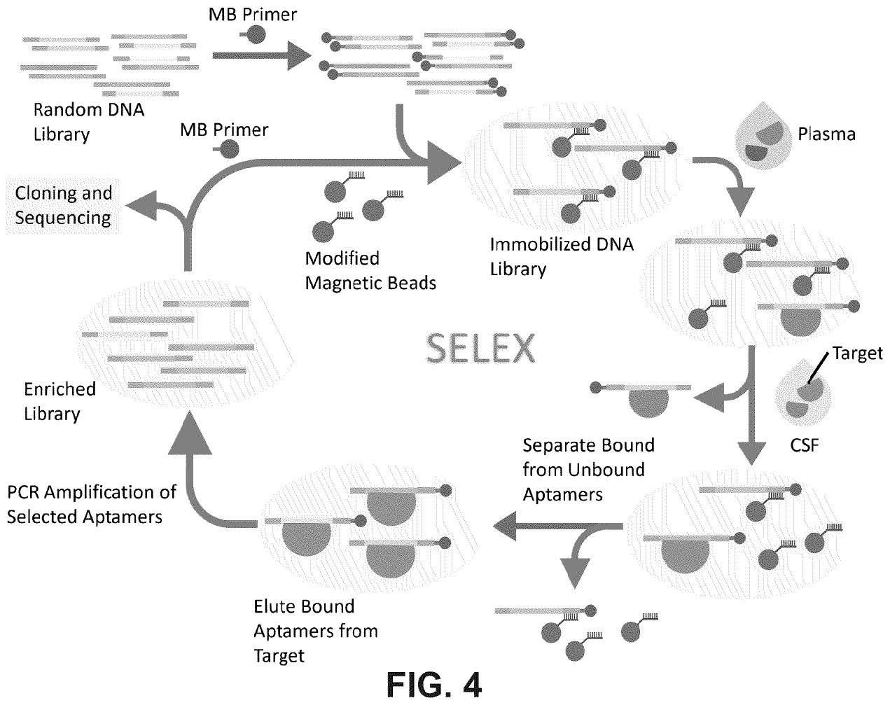

A pool of DNA sequences containing a 63-nucleotide (nt) stretch of random sequences flanked by two primer-binding sites was designed, as is typical for in vitro selection experiments (FIG. 4). The sequence diversity of the synthesized pool was approximately 1016. This ssDNA pool was further modified to contain a molecule of methylene blue (MB) at its 5′ terminus. The primers used to amplify the pool by polymerase chain reaction (PCR) and to make single-stranded variants of it also contained 5′ MB, so that in all rounds of the in vitro selection the ssDNA from which the CSF biomarker-binding aptamers are to be identified, all contained MB at the 5′ termini. MB is a redox indicator dye and electron donor that has previously been used in aptamer-based electrochemical systems, in which a designated target binding to the aptamer results in a conformational change that generates a change in electron transfer efficiency from MB to a gold substrate upon which the aptamer is anchored.

The design incorporated the electron donor into the selection pool such that any biomarker recognized by an aptamer arising from the pool has the opportunity to interact with the MB moiety. It was hypothesized that any such interaction would further strongly modulate the ability of the MB to donate an electron to the substrate, resulting in greater signal change due to ligand binding. A “capture oligonucleotide” (a biotinylated DNA oligo) was designed that base-pairs to the first 10 nts at the 5′-terminus of the pool. This was a key element of the selection, as the capture oligo is bound to streptavidin beads, and the pool is introduced at slightly elevated temperatures to facilitate the binding of the ssDNA pool to the capture oligo. The 10 base-pair interaction is strong enough to capture the pool at ambient temperatures, but weak enough that a conformational change that disrupts even 2 or 3 of the interacting nucleotides will result in the release of the sequence from the beads. Thus, the ssDNA pool was captured onto streptavidin beads, extensively washing the beads with phosphate-buffered saline (PBS) and plasma to remove sequences that are weakly bound or can respond to proteins (e.g., non-β2 transferrins) in human plasma, and then introduced pure CSF obtained from lumbar drains of patients undergoing skull base surgery. Of note, a different CSF specimen from a different patient was used in each round of selection to avoid bias toward a single clinical sample. Those DNA sequences collected and amplified were released in the presence of CSF, hypothesizing that they are enriched for ssDNA that bind CSF-specific biomarkers and undergo a conformational change substantial enough to result in dissociation from the capture oligo. This approach to select structure-switching DNA aptamers has been extensively used previously to identify target-binding aptamers with high potential for diagnostics.

The MB was incorporated into the pool so that the aptamers were selected in its presence and could interact with it. After 7 rounds of selection for MB-tagged ssDNA sequences that are released from the capture beads in the presence of CSF, the library and bioinformatic analysis were sequenced, which revealed several highly enriched sequences that were analyzed further via quantitative PCR (qPCR) and fluorescence anisotropy. The additional characterization will be performed via gel-shift and filter-binding assays and subsequently used for initial system construction.

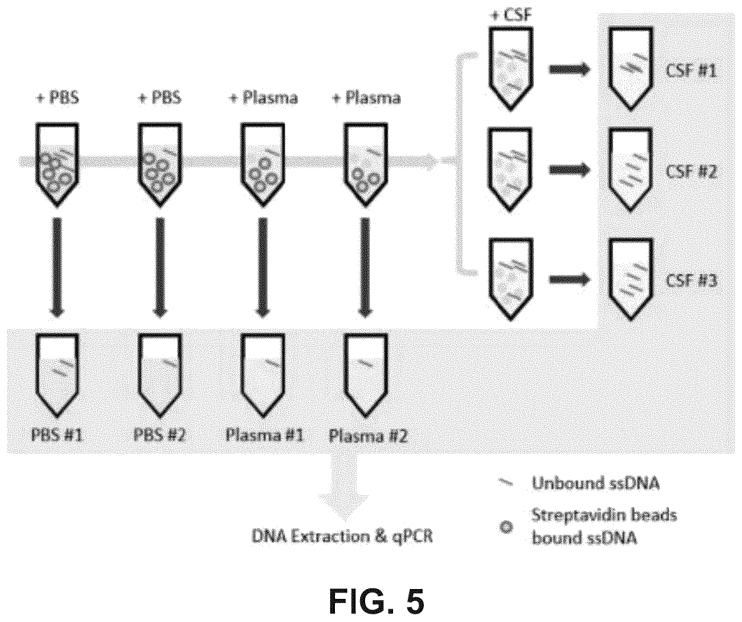

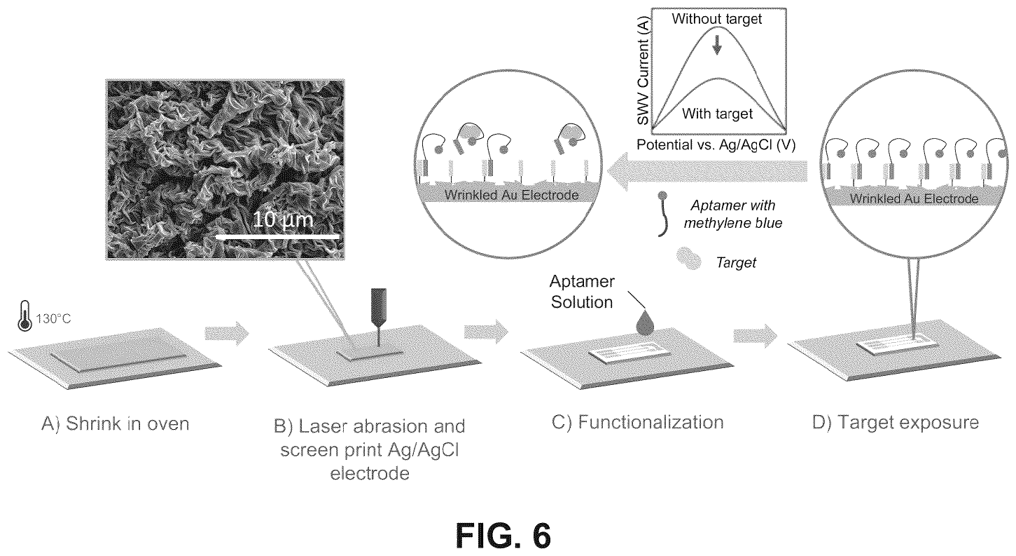

To evaluate the binding capacity and selectivity of the proposed aptamer, synthesized ssDNA (candidate aptamer) was processed using complex samples of plasma or CSF per the protocol outlined in FIG. 5A. The samples resulting from the PBS and plasma washes were analyzed using qPCR. It was found that the concentrations of the aptamer were significantly higher in the three CSF washes compared to the PBS and plasma washes. The low concentrations of aptamer detected after exposure to plasma indicated that the aptamer did not effectively “switch off” and unbind from the streptavidin beads in the presence of plasma-specific proteins (e.g., non-β2 transferrin, albumin, thrombin). Importantly, the concentrations of ssDNA following plasma washes were not significantly different from those after PBS washes, suggesting that the low levels of detected ssDNA may have been due to passive dissociation of the aptamer from the streptavidin beads, potentially exacerbated by turbulent mixing during the washes. On the other hand, the higher aptamer concentrations following exposure to CSF indicate that the aptamer was highly selective for a CSF-specific biomarker.

Fluorescence anisotropy (FA), wherein plane-polarized light is used to excite a fluorophore and the extent of depolarization of emitted fluorescence from rotational diffusion is measured, is frequently used to characterize aptamer-target binding. When unbound, fluorescently labeled aptamers exhibit rapid rotational diffusion and low anisotropy. When bound, the resulting aptamer-target complex undergoes slower rotational diffusion and emits greater depolarized light, which results in higher anisotropy. To evaluate the aptamer's binding capacity and its propensity for conformational change in the presence of the CSF biomarker, the FA of MB was measured in samples containing a constant aptamer concentration (1 μM) incubated for 20 minutes with varying concentrations of CSF. Of note, since CSF exhibits a similar viscosity to water, the FA measurements were unlikely to have been confounded by a change in sample viscosity.114 The FA of MB was measured in triplicates using a spectrofluorometer with excitation at 665 nm and emission at 700 nm. The findings demonstrated increasing FA with higher concentrations of CSF, suggesting target-induced conformational changes in the aptamer and a gradient response between the bound aptamer and free target protein concentration.

By using commodity ‘shrink-wrap films’ (pre-stressed thermoplastic), low-cost, scalable high-surface-area electrodes were created that leverage the stiffness-mismatch between the thin fold foil sputtered on the plastic. When the shrink film shrinks, it forces the metal to buckle or wrinkle; the resulting topography (FIG. 5) provides a >20-fold increase in surface area-to-volume ratio. An individual wrinkled (SDW) working electrode was successfully applied to E-AB detection of the SARS-COV-2 spike protein (S1) and demonstrated more reliable and significantly higher current densities than commercial disc electrodes.

To enable portable use for on-site clinical testing, an entire miniaturized electrochemical cell (SDW mini cell) consisting of wrinkled reference, counter, and working electrodes was fabricated on one substrate and interfaced with a Sensit® Smart (PalmSens, Houten, Netherlands) USB drive-sized potentiostat, which was capable of electrochemical measurements within 250 μL droplets. Compared to the individual SDW electrode, the SDW mini cell demonstrated as high as a two-fold increase in MB peak current densities and, on average, a 275% increase in signal gain at the highest concentration of spike protein. Importantly, the SDW mini cell demonstrated the detection of as little as 1 ag/mL S1 protein in 10% saliva, which is more than 12 orders of magnitude smaller than the concentration of β2TF in CSF. This suggested that the miniaturized system can be portable without sacrificing signal intensity. Moreover, leveraging aptamer kinetics can allow for response times on the order of seconds and, as demonstrated, provides equilibrium results and test-to-result times within minutes. Thus, the successful application of a compact design electrode system for high-sensitivity protein detection reinforces the feasibility of the proposed system to be used for on-site clinical testing for rapid point-of-care diagnosis of CSF leaks.

The following is another non-limiting example of the present invention. It is to be understood that said example is not intended to limit the present invention in any way. Equivalents or substitutes are within the scope of the present invention.

A single-stranded deoxyribonucleic acid (ssDNA) aptamer capable of selectively binding to CSF-specific biomarkers for the development of an aptamer-based POC CSF detection device, was developed. To identify a candidate aptamer, Systematic Evolution of Ligands by EXponential Enrichment (SELEX) using a DNA library containing a randomized 63-nucleotide (nt) stretch flanked by 2 primer-binding sites was performed. Quantitative polymerase chain reaction (qPCR) and fluorescence anisotropy (FA) assessed aptamer binding affinity and kinetics. Following 14 SELEX cycles, 2 dominant and functionally viable 98-nt ssDNA sequences (C2 and C3) were found. C2 and C3 demonstrated ˜586× and ˜82× higher affinity for CSF than plasma, respectively (both p<0.001). Increases in FA upon aptamer exposure to higher CSF concentrations demonstrated an apparent Ka of 5.0% and 14.1% CSF for C2 and C3, respectively. In-vitro selection of a diverse pool of ssDNA sequences yielded 2 aptamers with high selectivity for CSF-specific biomarkers, with potential for integration into a rapid POC electrochemical diagnostic system.

Cerebrospinal fluid (CSF) leak, which may occur anywhere along the skull base and emerge from the ear or nose, can be a result of a defect in the closed central nervous system due to trauma, surgical complication, spontaneous intracranial pressure elevation, or surgically created for skull base surgery (as well as failed reconstruction). Untreated CSF leak can lead to life-threatening consequences including meningitis, intracranial infection, or death, warranting prompt treatment that may range from conservative measures to urgent surgical treatment. There is mounting evidence to suggest that the incidence of CSF leaks is rising, partly due to the obesity epidemic and partly due to increased endoscopic endonasal surgery. CSF leaks are also associated with tremendous socioeconomic burden, namely additional diagnostic and therapeutic costs amounting to $15,000-$28,000 per patient. Therefore, prompt and accurate diagnosis of CSF leaks is critical to delivering high-quality patient care.

Unfortunately, distinguishing CSF leaks from physiologic nasal/otic secretions can be challenging, as patients commonly exhibit increased secretions after trauma or surgery. Furthermore, leaks secondary to elevated ICP can have delayed presentations due to resolving brain edema, devascularization of tissue, or formation of fistulae. For most clinicians, characterizing a colorless and odorless discharge such as CSF involves a high degree of clinical suspicion and may include a combination of history and physical examination, imaging, and laboratory testing. Traditionally, the “halo” or “ring” test has been suggested to provide diagnostic value; however, this has since been found to be highly nonspecific. While high-resolution computed tomography and magnetic resonance imaging are commonly acquired to aid with diagnosis, this can be time-consuming, costly, and even contraindicated in select patients, without certainty of diagnosis. Moreover, signs and symptoms of CSF leak are nonspecific and may include headache, tinnitus, malaise, fever, salty/metallic taste, or nasal/ear drainage, thereby risking misdiagnosis even by the most experienced clinicians. Conversely, exploration of a suspected CSF leak is more definitive, but if negative, is associated with surgical risks, risks of general anesthesia, and for postoperative cases, possible disruption of an intact repair or worsening pneumocephalus, as well as increased healthcare costs.

The current gold standard technique to diagnose suspected CSF leaks involves beta-2 transferrin (β2TF) immunofixation. Transferrin, a glycoprotein essential for iron homeostasis, is present in multiple forms within the human body. In the brain, its neuraminidase-eliminated terminal sialic acid residues on the glycan chain result in asialo-transferrin, also known as β2TF, which constitutes approximately one-third of total transferrin in the CSF.18 Among various biochemical assays used for detecting CSF, β2TF is regarded as the most reliable and specific biomarker. Its absence in physiologic nasal/otic secretions or blood makes it an ideal biomarker, hence its use in various techniques such as gel electrophoresis, immunofixation, or isoelectric focusing for detecting CSF with as high as 100% sensitivity and 94% specificity, though false negative results are certainly possible. Current state-of-the-art technology allows for accurate β2TF detection via electrophoresis; however, this technique's accessibility and practicality have been largely hindered by its 3-7 day latency time and need for sample purity, adequate sample quantity, and skilled professionals at a specialized laboratory. As a send-out test at designated laboratories and centers, capabilities to test for β2TF may not be readily available in many lower-resource areas, such as rural centers, which can impact care.

Electrochemical sensors are recognized for their high sensitivity, selectivity, and rapid response time. They detect trace amounts of substances, differentiate between compounds, and offer real-time data for swift reactions to changes. Their portability, low power consumption, and long-term stability make them ideal for medical diagnostics. Electrochemical aptamer-based (EAB) sensors offer a unique advantage due to the specificity and sensitivity of aptamers. Aptamers are single-stranded DNA or RNA molecules that bind to specific target molecules with high affinity, akin to antibodies. Aptamers are identified through their highly selective interaction with proteins using an approach known as Systematic Evolution of Ligands by EXponential enrichment (SELEX) or in vitro selection—starting with a target protein with known function, a large and diverse random sequence deoxyribonucleic acid (DNA) library is screened for specific interaction. Several in vitro selections have yielded aptamers that specifically bind a single conformation of their target proteins, including the ATP-bound form of the Hsp70 chaperone, Ca²⁺-bound form of calsenilin, and β-form of the prion protein. When integrated into electrochemical sensors, aptamers enhance the device's selectivity, allowing for precise detection of target molecules. This specificity, combined with the electrochemical sensing mechanism, results in sensors with rapid response times and exceptional sensitivity, capable of detecting trace amounts of the target molecule.

To date, there exists no aptamer specific to CSF-specific biomarkers. With the goal of developing an EAB sensor for POC CSF leak detection, a novel aptamer with high binding affinity and specificity for CSF-specific biomarkers was sought. Specifically, a conformation-switching aptamer, which would not only bind to its target but also change conformation upon binding was developed. This is a desirable property for integration with an EAB sensor since when exposed to the target molecule, the aptamer can undergo a conformational change (“switch”) that can be coupled with a measurable change in conductance. Thus, in vitro selection of DNA conformation-switching aptamers was implemented to recognize biomarkers in CSF. For the purposes of an EAB sensor, oligonucleotides were modified with a methylene blue (MB), a redox indicator, at their 5′ termini to be incorporated during the selection process. The in vitro selected pool of DNA aptamers went through 14 rounds of selection in the presence of pooled patient CSF samples. The most enriched sequences, C2 and C3, were evaluated for their binding affinity and specificity for CSF-specific biomarkers.

Streptavidin magnetic beads (Pierce™) and 10× DPBS were purchased from Thermo Fisher Scientific™ (Waltham, MA, USA). Amicon® ultra centrifugal filter (50 kDa), phenol solution, Triton™ X-100 were purchased from Sigma-Aldrich® (St. Louis, MO, USA). The DNA library (Polyacrylamide gel electrophoresis (PAGE) purification), methylene blue (MB) primer (HPLC purification), and the biotinylated capture probes were purchased through Integrated DNA Technologies® (IDT®; Coralville, IA, USA). The DNA library contains a 63-nucleotide randomized region flanked by the primer binding sequences for the selection.

The sequences utilized are as follows:

| DNA Library | 5′-actcgacgcgctgtccc-n63-ggtgccgtaag |
|---|---|
| | tgatctccc-3′ (SEQ ID NO: 7) |
| Capture | /5Biosg//iSp18//iSp18//iSp18/ |
| probe | cgcgtcgagt-3′ (SEQ ID NO: 8) |
| MB primer | /5MeBIN/actcgacgcgctgtccc-3′ (SEQ ID |
| | NO: 9) |
| Cy3 primer | /5Cy3/actcgacgcgctgtccc-3′ (SEQ ID |
| | NO: 10) |
| Forward | 5′-gggagatcacttacggcacc-3′ (SEQ ID |
| primer | NO: 11) |
| Reverse | 5′-cgacgcgctgtcccg-3′ (SEQ ID NO: |
| primer | 12) |

10 μL of streptavidin magnetic beads was washed three times with 200 μL of 1× DPBS with 0.1% of Triton™ X-100 (Sigma-Aldrich®), and resuspended with 1 μM of biotinylated capture probe. After 5-min incubation using gentle rotation, the liquid was removed, and beads were resuspended in 60 μL of DPBS.

To generate the ssDNA library, the MB primer was used to amplify the starting DNA library using a final concentration of 1× Taq Reaction Buffer, 0.2 mM dNTPs each, 2 μM MB primer, and 1.25 U of DNA Taq Polymerase. The reaction was amplified for 10 to 16 cycles (95° C. for 30 s, 55° C. for 30 s, and 72° C. for 30 s), to determine the optimal amplification. A 3% agarose gel electrophoresis (Sigma-Aldrich®) was used to visualize the amplification product for each round. The MB-modified library (2 μL) was hybridized with the capture probe on the magnetic beads by incubating for 5 mins with gentle rotation. The liquid was removed and beads with the hybridized library were resuspended in 200 μL of 1× DPBS for selection.

The magnetic beads with the hybridized library were washed twice with 200 μL of 1× DPBS prior to the counter selection. For the first to fifth rounds, a final concentration of 1% pooled human plasma (Innovative Research™, Los Angeles, CA, USA) with 2 mM EDTA (Thermo Fisher Scientific™) and 1× DPBS was added into the beads as a counter selection. From the sixth to the ninth round, 1% pooled human serum (Innovative Research™) with 1× DPBS was used as the counter selection, and from the tenth to the thirteen round, 50% serum was used as the counter. Finally, The fourteenth round of selection included a 100% serum as the counter. Serum elutions from rounds 10, 13, and 14 were collected for next-gen sequencing (NGS) analysis. After the counter-selection step, a wash with 1× DPBS was performed prior to eluting DNA in the presence of CSF. CSF was then added to the beads and incubated for 5 minutes using gentle rotation to elute DNA from the magnetic beads. CSF fractions were collected and a phenol-chloroform extraction was performed to separate ssDNA prior to precipitation with 300 mM KCl, 1 μl Glyco-Blue™, and 2.5 volumes of cold 100% ethanol at −20° C. The ssDNA was further purified using a 50 kDa Amicon® filter to remove any water-soluble protein in the sample and further precipitated as described above.

The purified ssDNA was PCR amplified prior to the next round. The PCR reaction consisted of 1× Standard Taq Reaction Buffer, 0.2 mM dNTPs each, 1 μM of both the forward and reverse primers, ssDNA, and 1.25 U Taq polymerase. The ssDNA was amplified similarly to the described method above except the annealing temperature was set to 65ºC for 30 seconds. A 3% agarose gel electrophoresis was used to check the amplified DNA prior to the beginning of each selection round.

The CSF elution from rounds 7-10, 13, and 14 as well as the countered serum elutions from rounds 10, 13, and 14 were prepared for Illumina® MiSeq™ sequencing (Illumina® Inc., San Diego, CA, USA). DNA was amplified using the TruSeq® forward and reverse adapter in a final reaction containing 1× Standard Taq Reaction Buffer, 0.2 mM of dNTPs, 1 μM of each primer, and 1.25 U DNA Taq polymerase. The reactions were amplified for one cycle (95° C. for 30 s, 65° C. for 30 s, 72° C. for 30 s, followed by multiple repeated cycles (95° ° C. for 30 s, 70° C. for 30 s, and 72° C. for 30 s) until desired products were visible. Primer amplification was confirmed using a 3% gel electrophoresis prior to purifying DNA with a Zymo™ DNA Gel Extraction Kit. DNA was then submitted to next-generation sequencing (NGS).

Sequenced reads were obtained using an Illumina® MiSeq™. After, reads were merged with PEAR using default settings and Bowtie2 was used to remove any reads matching the PhiX genome. FASTAptamer was used to count and then cluster sequences with an edit distance of 10. Candidate aptamers were identified from the enrichment of sequences throughout multiple selection rounds and then characterized for binding to CSF.

The DNA constructs were purchased from IDT® and the MB primer was used to amplify the ssDNA aptamer for hybridization to the capture oligo. The beads were washed with 1× DPBS with 0.1% Triton™ X-100 and all washes were collected for further analysis. After, 7M UREA with 40 mM EDTA was added to the streptavidin beads, and the suspension was heated at 95ºC for 2 minutes to remove any remaining DNA. After, DNA from all six washes was phenol-chloroform extracted and purified using a 50 kDa Amicon® filter. Quantitative PCR (qPCR) was used to assess the amount of DNA that was present within each fraction. All qPCR reactions were performed with a final concentration of 1× iTaq Universal SYBR® Green Super mix (Bio-Rad® laboratories), 1 μM forward, and reverse primers. The amplification reactions were performed on a CFX Connect™ Real-Time PCR (Bio-Rad® laboratories). Cq values were subsequently determined for each fraction and a standard curve was used to calculate the amount of DNA in each sample.

Fluorescence anisotropy was performed using aptamers containing a Cy3 on the 5′ end. Each individual construct was amplified with a Cy3 primer using the same amplification protocol from the selection. The fluorescence experiment was performed using a spectrofluorometer (JASCO® FP-6300, Jasco® Inc., Easton, MD, USA). Excitation and emission wavelengths used for Cy3 were 545 and 565 nm, respectively. Fluorescent intensities were measured with the combination of the Cy3 ssDNA amplicon and diluted CSF in the final percentages of 1%, 3%, 5%, 10%, 20%, 40%, 60%, 87%. Since CSF possesses a similar viscosity to water, FA measurements were unlikely to have been confounded by a change in sample viscosity at different CSF concentrations.39 Three data points were collected for all anisotropy measurements and the averaged numbers were used for data processing. The anisotropy r of Cy3 labeled DNA was calculated based on the following equation:

$$r = \frac{{I}_{VV} - {GI}_{VH}}{{I}_{VV} + 2 {GI}_{VH}} where G = \frac{{I}_{HV}}{{I}_{HH}}$$

Where I represented the fluorescence intensity and the subscripts represented the orientation of the polarizers at the emission and detection channel, respectively. Kₐ was determined by fitting the data to the Michaelis-Menten equation using non-linear (weighted) least-squares regression in R (version 3.6.1; The R Foundation for Statistical Computing).

Magnetic beads (48 μL) were washed and immobilized with capture oligos following the immobilization protocol. A single sequence of the MB amplicon was hybridized to the beads following the protocol described above. Then the beads were washed four times in DPBS before being aliquoted into 8 portions. Diluted CSF with DPBS was added into each portion of the beads in the following percentages: 0%, 1%, 2.5%, 10%, 20%, 40%, 50%, 75%. Beads were incubated for 10 minutes in gentle rotation before the diluted CSF was obtained for further extraction and purification. qPCR was performed following the protocol described above. The secondary structures of the aptamers were predicted using the online software oligoAnalyzer™ 3.1 (or RNAfold). The simulations were done assuming ionic conditions of Na+(mM) and Mg2+(mM) at ºC.

A pool of DNA sequences containing a 63-nucleotide (nt) stretch of random sequences flanked by two primer-binding sites was designed. The sequence diversity of the synthesized pool was approximately 1016. This ssDNA pool was further modified to contain a molecule of methylene blue (MB) at its 5′ terminus (FIG. 4). The primers used to amplify the pool by PCR and to make single-stranded variants of it also contained 5′ MB, so that in all rounds of the in vitro selection the ssDNA from which the CSF biomarker-binding aptamers were to be identified, all contained MB at the 5′ termini. A “capture oligonucleotide” (a biotinylated DNA oligo) that base-paired to the first 10 nucleotides (nts) at the 5′-terminus of the pool was designed. This was a key element of the selection, as the capture oligo was bound to streptavidin beads, and the pool was introduced to facilitate the binding of the ssDNA pool to the capture oligo. The 10 base-pair interaction was strong enough to capture the pool at ambient temperatures, but weak enough that a conformational change that disrupted even 2 or 3 of the interacting nucleotides would result in the release of the sequence from the beads. Thus, the ssDNA pool was captured onto streptavidin beads, extensively washing the beads with phosphate-buffered saline (PBS) and plasma to remove sequences that were weakly bound or could respond to proteins (e.g., albumin) in human plasma, and then introduced pure CSF obtained from lumbar drains of patients undergoing skull base surgery. Those DNA sequences released in the presence of CSF, were collected and analyzed. It was hypothesized that they were enriched for ssDNAs that bind CSF-specific biomarkers and undergo a conformational change substantial enough to result in dissociation from the capture oligonucleotide.

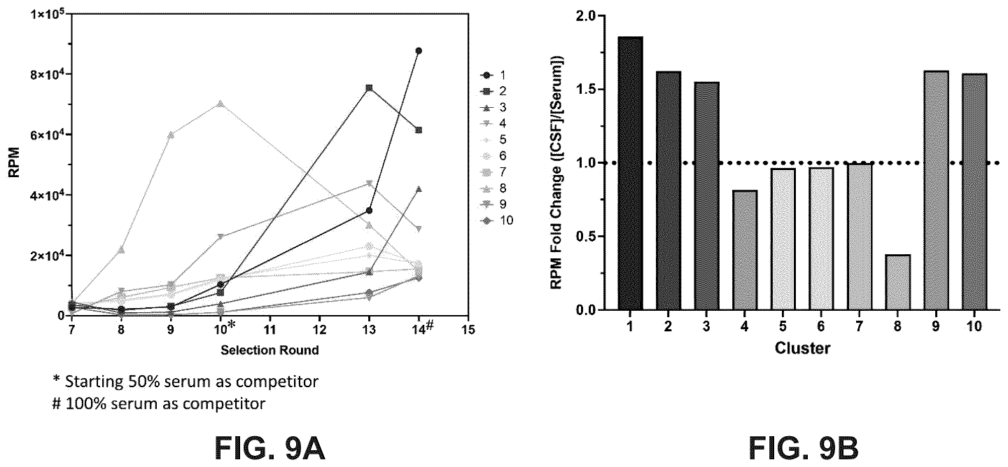

A total of 14 rounds of SELEX were performed to identify candidate CSF-specific aptamers. With the initial goal of identifying CSF biomarker-specific binders, either 5% BSA or 1% serum were used as the counterselection agents for the first 9 rounds of in vitro selection. For subsequent cycles (#10-14), high serum concentrations were used with the goal of delineating candidate aptamers that possess high specificity for CSF and discrimination against physiological contaminants present in serum. After 14 rounds of selection, the library was sequenced and bioinformatic analysis revealed several highly enriched sequences (FIGS. 9A-9B). In identifying candidate aptamers, two criteria were considered: (1) high sequence enrichment (i.e., reads per million [RPM]) and (2) greater sequence enrichment in CSF relative to serum. Among the top 10 sequence clusters, only the first 3 clusters (C1, C2, C3) met both criteria and were further characterized with quantitative PCR (qPCR).

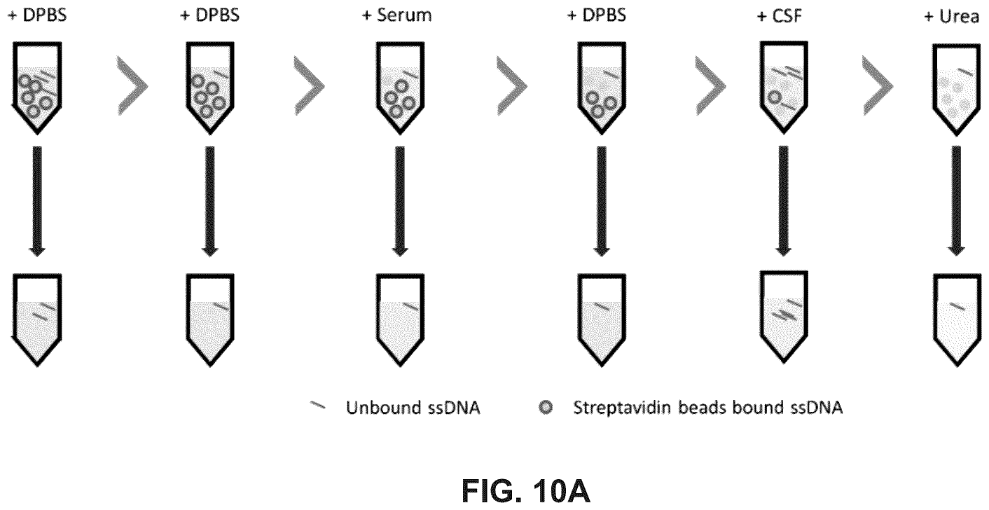

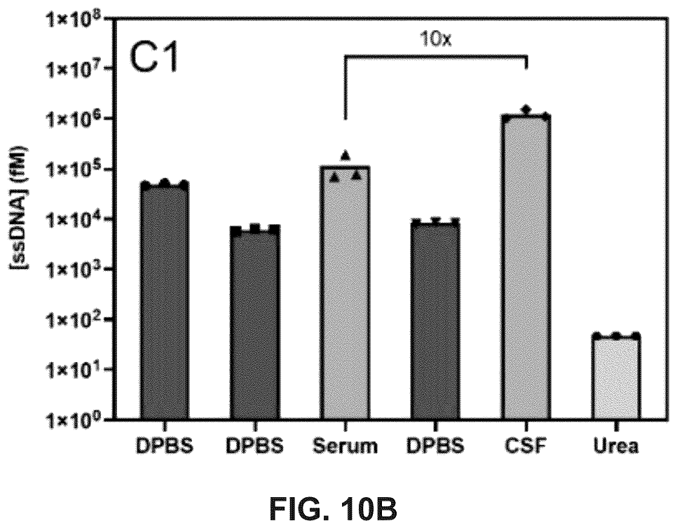

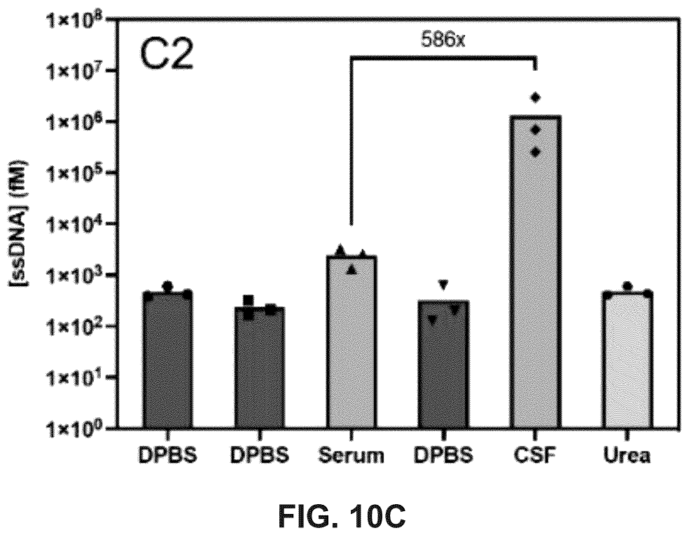

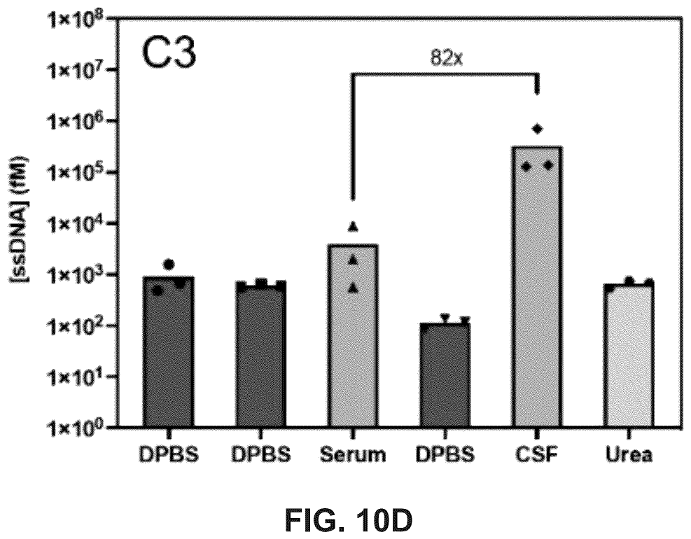

To evaluate the binding capacity and selectivity of the candidate aptamers (C1-C3), the streptavidin bead-bound ssDNA were sequentially incubated in PBS, serum, and CSF per the protocol outlined in FIGS. 10A-10D. ssDNA that interacted with molecules in the solutions would unbind from the beads and fall into the solution. Thus, by using qPCR on the resulting solution washes, the amount of ssDNA that bound to the molecules in each solution was quantified. The PBS washes served as negative controls, and ssDNA that unbound due to these washes were likely due to passive dissociation from turbulent mixing and indicated poor binding strength. C1 and C2 demonstrated the highest ssDNA concentration in CSF. However, C1 exhibited relatively high ssDNA concentrations in serum, suggesting poor specificity for CSF. Therefore, when considering both binding affinity and specificity, qPCR results favored C2 and C3 as candidate aptamer sequences for further analysis.

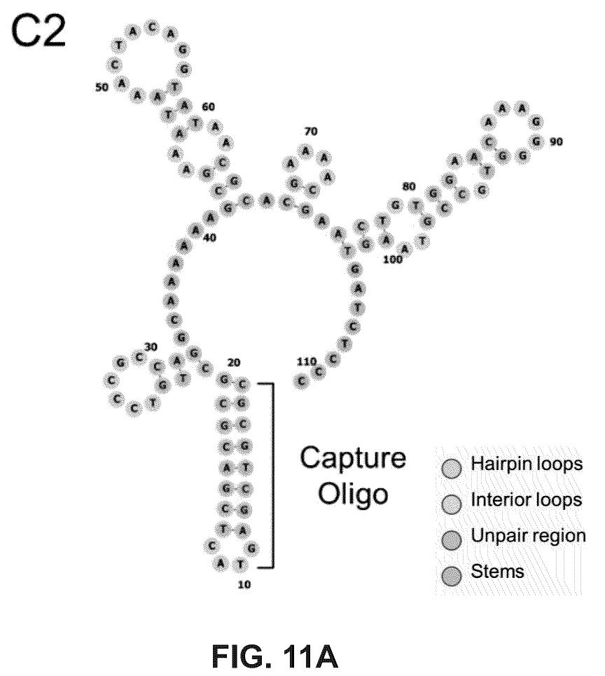

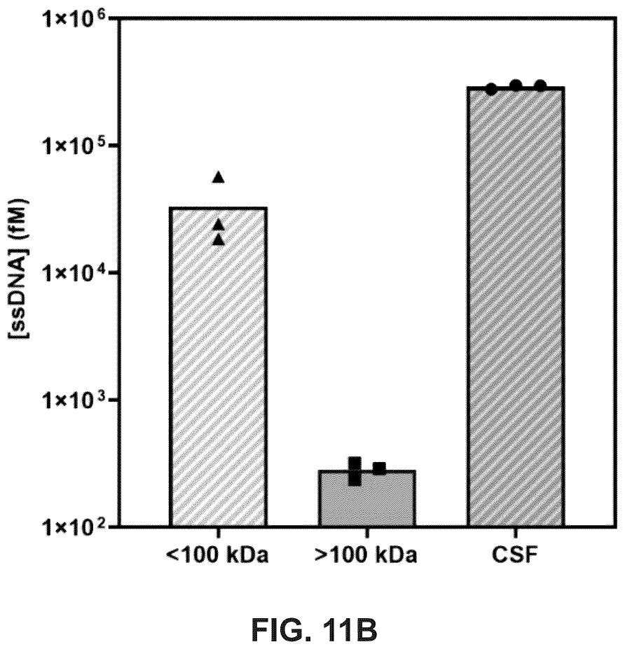

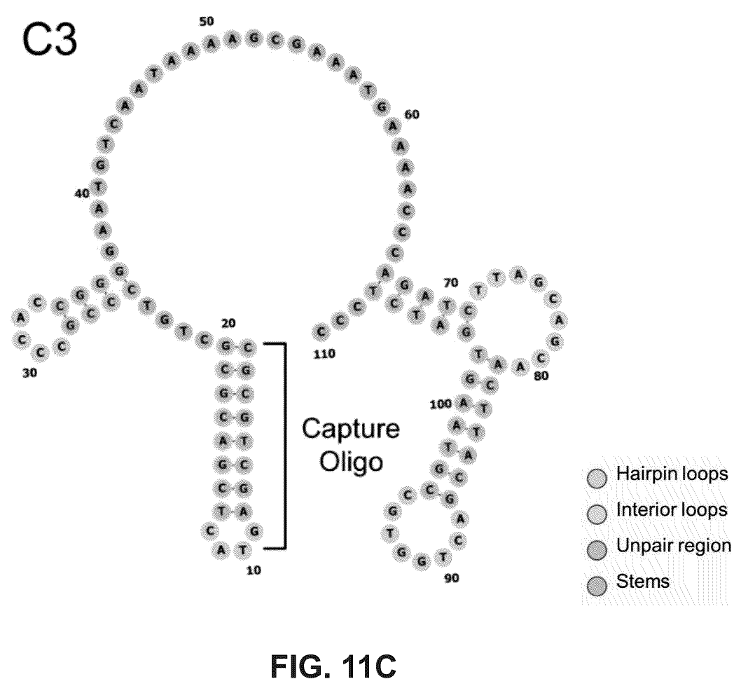

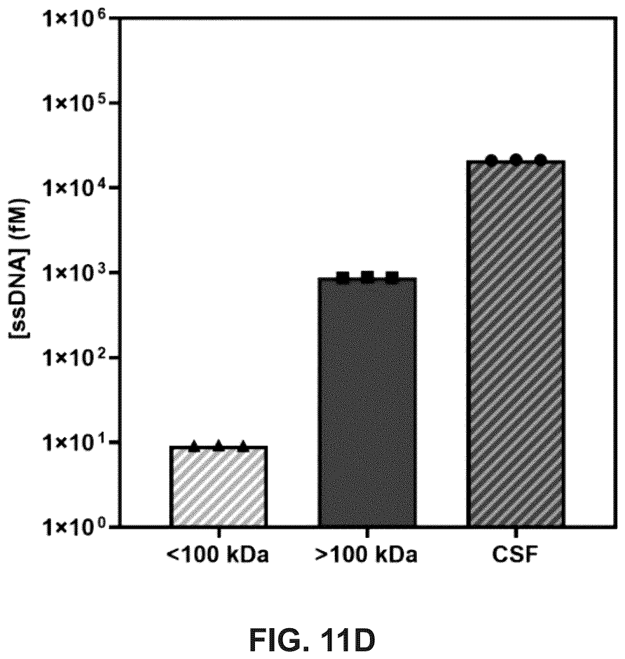

Secondary structures for C2 and C3 sequences were predicted using RNAfold (FIGS. 11A-11D). Due to differences in their predicted structures, it was hypothesized that C2 and C3 may also bind to different target molecules. Using size exclusion filtration, the whole CSF was fractionated into solutions consisting of molecules <100 kDa and >100 kDa in size. The candidate aptamers were then tested using samples of whole CSF, <100 kDa CSF, and >100 kDa CSF per the protocol in FIGS. 10A-10D. qPCR analysis suggested that C2 may favor molecules of <100 kDa while C3 may favor molecules of >100 kDa (FIGS. 11A-11D).

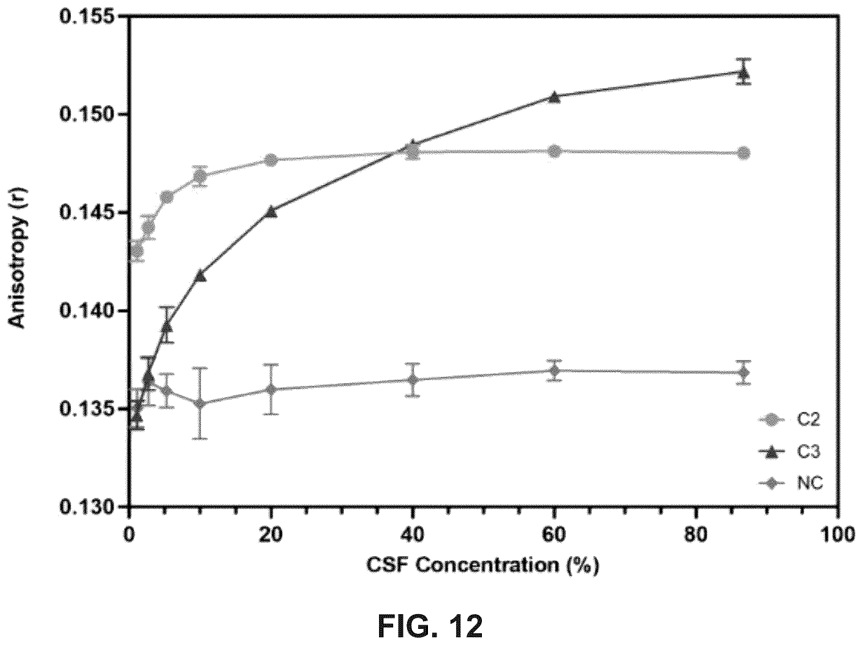

Fluorescence anisotropy (FA), wherein plane-polarized light is used to excite a fluorophore and the extent of depolarization of emitted fluorescence from rotational diffusion is measured, was used to characterize aptamer-target binding. When bound to its target, the aptamer is expected to undergo slower rotational diffusion and emit greater depolarized light, resulting in higher anisotropy. Thus, to evaluate the aptamers' binding capacities and their propensity for conformational change in the presence of the target CSF biomarkers, the FA in samples containing a constant aptamer concentration incubated with varying concentrations of CSF was measured (FIG. 12). A random 98-nt sequence containing the same 5′-terminus fluorophore and primer sequence was used as the negative control (NC). C2 and C3 demonstrated increasing anisotropy with higher concentrations of CSF, while the NC did not exhibit any significant anisotropy changes. Applying nonlinear regression on the data per the Langmuir Isotherm equation demonstrated apparent Ka values of 5.0% and 14.1% CSF for C2 and C3, respectively.

SELEX was utilized to discover conformation-switching DNA aptamers that could bind to CSF-specific biomarkers and discriminate against contaminating molecules from other physiological fluids (e.g., serum). After 14 cycles of in vitro selection, two 98-nt ssDNA sequences were identified that demonstrated favorable binding kinetics and selectivity for CSF compared to serum. Although recent efforts in CSF detection have implemented immunoassays, aptamers have been shown to possess significant advantages over antibodies due to their nucleic acid nature and target-induced structure-switching properties. As they are chemically synthesized through an in vitro process, aptamers not only offer remarkable design flexibility but are inexpensive to produce and are generated to a high degree of purity with little to no batch-to-batch variation.

When developing an aptamer to be implemented for CSF detection, in addition to binding affinity, strength of binding and specificity for CSF biomarkers were critical. In the qPCR analysis, the concentrations of C2 and C3 were found to be significantly higher in the CSF wash compared to the DPBS and serum washes. The low aptamer concentrations detected after exposure to serum indicated that the aptamer did not effectively “switch off” and unbind from the streptavidin beads in the presence of serum-specific proteins (e.g., transferrin, albumin, thrombin). Importantly, the concentrations of ssDNA following serum washes were not significantly different from those after DPBS washes, suggesting that the low levels of detected ssDNA may have been due to passive dissociation of the aptamers from the streptavidin beads, potentially exacerbated by turbulent mixing during the washes. On the other hand, the higher aptamer concentrations following exposure to CSF indicated that the aptamers were highly selective for CSF-specific biomarkers. Additionally, low concentrations of ssDNA were found remaining on the streptavidin beads after CSF exposure, indicating that a low volume of CSF contained a sufficient concentration of target molecules to interact with and induce a conformational switch in most of the aptamers originally bound to streptavidin beads.

The dose-dependent rise in FA indicated an increase in the concentration of aptamer-target complexes, thereby supporting that C2 and C3 undergo a conformational change when bound to CSF-specific biomarkers. This is an important characteristic for future implementation into an E-AB sensor, since a conformational switch and, hence, positional change in the MB moiety will be required to induce a detectable change in signal. Within the framework of an electrochemical sensor, MB's role as an electron donor becomes crucial. As this altered MB moiety comes into proximity with the gold substrate of the sensor to which the aptamer is affixed, it induces a notable change in the efficiency of electron transfer. This alteration is detectable as a change in electrical current, forming the basis for detecting the presence of CSF-specific biomarkers in an E-AB sensor's readout.

Interestingly, experiments using size-excluded CSF samples suggested that the aptamers may have a tendency to interact with different targets. Specifically, C2 appeared to favor molecules <100 kDa in size while C3 favored molecules larger than 100 kDa. These findings are in line with observations from FA analysis, which demonstrated a greater rise in anisotropy with aptamer-target complexes of C3 compared to C2. Since C2 and C3 are the same sizes, a greater change in anisotropy following target binding suggests a larger-sized target. Although the identities of the aptamers' target molecules are not known, given that the estimated size of (2TF is ˜78 kDa and beta-trace protein is 23-29 kDa, it is possible that C2 is interacting with one of these well-established CSF biomarkers. However, it is unclear if the target of C3, which exceeds 100 kDa, is an oligomer of β2TF or beta-trace protein or if it is a larger molecule that has yet to be established as a distinctive biomarker of CSF.

14 cycles of in vitro selection were conducted to uncover two conformation-switching 98-nt ssDNA aptamers capable of selectively binding to CSF-specific biomarkers. qPCR and FA analysis demonstrated favorable binding kinetics for CSF detection and strong discrimination against similar molecules found in human serum. Integration of these CSF-specific aptamers into an E-AB sensor may serve as an accurate and cost-effective diagnostic modality for rapidly detecting CSF leaks.

The computer system can include a desktop computer, a workstation computer, a laptop computer, a netbook computer, a tablet, a handheld computer (including a smartphone), a server, a supercomputer, a wearable computer (including a SmartWatch™), or the like and can include digital electronic circuitry, firmware, hardware, memory, a computer storage medium, a computer program, a processor (including a programmed processor), an imaging apparatus, wired/wireless communication components, or the like. The computing system may include a desktop computer with a screen, a tower, and components to connect the two. The tower can store digital images, numerical data, text data, or any other kind of data in binary form, hexadecimal form, octal form, or any other data format in the memory component. The data/images can also be stored in a server communicatively coupled to the computer system. The images can also be divided into a matrix of pixels, known as a bitmap that indicates a color for each pixel along the horizontal axis and the vertical axis. The pixels can include a digital value of one or more bits, defined by the bit depth. Each pixel may comprise three values, each value corresponding to a major color component (red, green, and blue). A size of each pixel in data can range from a 8 bits to 24 bits. The network or a direct connection interconnects the imaging apparatus and the computer system.

The term “processor” encompasses all kinds of apparatus, devices, and machines for processing data, including by way of example a programmable microprocessor, a microcontroller comprising a microprocessor and a memory component, an embedded processor, a digital signal processor, a media processor, a computer, a system on a chip, or multiple ones, or combinations, of the foregoing. The apparatus can include special-purpose logic circuitry, e.g., an FPGA (field programmable gate array) or an ASIC (application-specific integrated circuit). Logic circuitry may comprise multiplexers, registers, arithmetic logic units (ALUs), computer memory, look-up tables, flip-flops (FF), wires, input blocks, output blocks, read-only memory, randomly accessible memory, electronically-erasable programmable read-only memory, flash memory, discrete gate or transistor logic, discrete hardware components, or any combination thereof. The apparatus also can include, in addition to hardware, code that creates an execution environment for the computer program in question, e.g., code that constitutes processor firmware, a protocol stack, a database management system, an operating system, a cross-platform runtime environment, a virtual machine, or a combination of one or more of them. The apparatus and execution environment can realize various different computing model infrastructures, such as web services, distributed computing and grid computing infrastructures. The processor may include one or more processors of any type, such as central processing units (CPUs), graphics processing units (GPUs), special-purpose signal or image processors, field-programmable gate arrays (FPGAs), tensor processing units (TPUs), and so forth.

A computer program (also known as a program, software, software application, script, or code) can be written in any form of programming language, including compiled or interpreted languages, declarative or procedural languages, and it can be deployed in any form, including as a stand-alone program or as a module, component, subroutine, object, or other unit suitable for use in a computing environment. A computer program may, but need not, correspond to a file in a file system. A program can be stored in a portion of a file that holds other programs or data (e.g., one or more scripts stored in a markup language document), in a single file dedicated to the program in question, or in multiple coordinated files (e.g., files that store one or more modules, subprograms, or portions of code). A computer program can be deployed to be executed on one computer or on multiple computers that are located at one site or distributed across multiple sites and interconnected by a communication network.

Embodiments of the subject matter and the operations described herein can be implemented in digital electronic circuitry, or in computer software, firmware, or hardware, including the structures disclosed in this specification and their structural equivalents, or in combinations of one or more of them. Embodiments of the subject matter described in this specification can be implemented as one or more computer programs, i.e., one or more modules of computer program instructions, encoded on computer storage medium for execution by, or to control the operation of, a data processing apparatus.

A computer storage medium can be, or can be included in, a computer-readable storage device, a computer-readable storage substrate, a random or serial access memory array or device, or a combination of one or more of them. Moreover, while a computer storage medium is not a propagated signal, a computer storage medium can be a source or destination of computer program instructions encoded in an artificially generated propagated signal. The computer storage medium can also be, or can be included in, one or more separate physical components or media (e.g., multiple CDs, drives, or other storage devices). The operations described in this specification can be implemented as operations performed by a data processing apparatus on data stored on one or more computer-readable storage devices or received from other sources.

Program code embodied on a computer readable medium may be transmitted using any appropriate medium, including but not limited to wireless, wireline, optical fiber cable, R.F, Bluetooth, storage media, computer buses, etc., or any suitable combination of the foregoing. Computer program code for carrying out operations for aspects of the present disclosure may be written in any combination of one or more programming languages, including an object oriented programming language such as Java, Smalltalk, C #, Ruby, or the like, conventional procedural programming languages, such as Pascal, FORTRAN, BASIC, or similar programming languages, programming languages that have both object-oriented and procedural aspects, such as the “C” programming language, C++, Python, or the like, conventional functional programming languages such as Scheme, Common Lisp, Elixir, or the like, conventional scripting programming languages such as PHP, Perl, Javascript, or the like, or conventional logic programming languages such as PROLOG, ASAP, Datalog, or the like.

The program code may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider).

The processes and logic flows described in this specification can be performed by one or more programmable processors executing one or more computer programs to perform actions by operating on input data and generating output. The processes and logic flows can also be performed by, and apparatus can also be implemented as, special purpose logic circuitry, e.g., an FPGA (field programmable gate array) or an ASIC (application-specific integrated circuit).

Processors suitable for the execution of a computer program include, by way of example, both general and special purpose microprocessors, and any one or more processors of any kind of digital computer. Generally, a processor will receive instructions and data from a read-only memory or a random access memory or both. The essential elements of a computer are a processor for performing actions in accordance with instructions and one or more memory devices for storing instructions and data. Generally, a computer will also include, or be operatively coupled to receive data from or transfer data to, or both, one or more mass storage devices for storing data, e.g., magnetic, magneto-optical disks, or optical disks.

However, a computer need not have such devices. Moreover, a computer can be embedded in another device, e.g., a mobile telephone, a personal digital assistant (PDA), a mobile audio or video player, a game console, a Global Positioning System (GPS) receiver, or a portable storage device (e.g., a universal serial bus (USB) flash drive), to name just a few. Devices suitable for storing computer program instructions and data include all forms of non-volatile memory, media and memory devices, including by way of example semiconductor memory devices, e.g., EPROM, EEPROM, and flash memory devices; magnetic disks, e.g., internal hard disks or removable disks; magneto-optical disks; and CD-ROM and DVD-ROM disks. The processor and the memory can be supplemented by, or incorporated in, special purpose logic circuitry.

Computers typically include known components, such as a processor, an operating system, system memory, memory storage devices, input-output controllers, input-output devices, and display devices. It will also be understood by those of ordinary skill in the relevant art that there are many possible configurations and components of a computer and may also include cache memory, a data backup unit, and many other devices. To provide for interaction with a user, embodiments of the subject matter described in this specification can be implemented on a computer having a display device, e.g., an LCD (liquid crystal display), LED (light emitting diode) display, or OLED (organic light emitting diode) display, for displaying information to the user.

Examples of input devices include a keyboard, cursor control devices (e.g., a mouse or a trackball), a microphone, a scanner, and so forth, wherein the user can provide input to the computer. Other kinds of devices can be used to provide for interaction with a user as well; for example, feedback provided to the user can be in any form of sensory feedback, e.g., visual feedback, auditory feedback, or tactile feedback; and input from the user can be received in any form, including acoustic, speech, or tactile input. Examples of output devices include a display device (e.g., a monitor or projector), speakers, a printer, a network card, and so forth. Display devices may include display devices that provide visual information, this information typically may be logically and/or physically organized as an array of pixels. In addition, a computer can interact with a user by sending documents to and receiving documents from a device that is used by the user; for example, by sending web pages to a web browser on a user's client device in response to requests received from the web browser.

An interface controller may also be included that may comprise any of a variety of known or future software programs for providing input and output interfaces. For example, interfaces may include what are generally referred to as “Graphical User Interfaces” (often referred to as GUI's) that provide one or more graphical representations to a user. Interfaces are typically enabled to accept user inputs using means of selection or input known to those of ordinary skill in the related art. In some implementations, the interface may be a touch screen that can be used to display information and receive input from a user. In the same or alternative embodiments, applications on a computer may employ an interface that includes what are referred to as “command line interfaces” (often referred to as CLI's). CLI's typically provide a text based interaction between an application and a user. Typically, command line interfaces present output and receive input as lines of text through display devices. For example, some implementations may include what are referred to as a “shell” such as Unix Shells known to those of ordinary skill in the related art, or Microsoft® Windows Powershell that employs object-oriented type programming architectures such as the Microsoft® .NET framework.

Those of ordinary skill in the related art will appreciate that interfaces may include one or more GUI's, CLI's or a combination thereof. A processor may include a commercially available processor such as a Celeron, Core, or Pentium processor made by Intel Corporation®, a SPARC processor made by Sun Microsystems®, an Athlon, Sempron, Phenom, or Opteron processor made by AMD Corporation®, or it may be one of other processors that are or will become available. Some embodiments of a processor may include what is referred to as multi-core processor and/or be enabled to employ parallel processing technology in a single or multi-core configuration. For example, a multi-core architecture typically comprises two or more processor “execution cores”. In the present example, each execution core may perform as an independent processor that enables parallel execution of multiple threads. In addition, those of ordinary skill in the related field will appreciate that a processor may be configured in what is generally referred to as 32 or 64 bit architectures, or other architectural configurations now known or that may be developed in the future.

A processor typically executes an operating system, which may be, for example, a Windows type operating system from the Microsoft Corporation®; the Mac OS X operating system from Apple Computer Corp.®; a Unix® or Linux®-type operating system available from many vendors or what is referred to as an open source; another or a future operating system; or some combination thereof. An operating system interfaces with firmware and hardware in a well-known manner, and facilitates the processor in coordinating and executing the functions of various computer programs that may be written in a variety of programming languages. An operating system, typically in cooperation with a processor, coordinates and executes functions of the other components of a computer. An operating system also provides scheduling, input-output control, file and data management, memory management, and communication control and related services, all in accordance with known techniques.

Connecting components may be properly termed as computer-readable media. For example, if code or data is transmitted from a website, server, or other remote source using a coaxial cable, fiber optic cable, twisted pair, digital subscriber line (DSL), or wireless technology such as infrared, radio, or microwave signals, then the coaxial cable, fiber optic cable, twisted pair, DSL, or wireless technology are included in the definition of medium. Combinations of media are also included within the scope of computer-readable media.

Although there has been shown and described the preferred embodiment of the present invention, it will be readily apparent to those skilled in the art that modifications may be made thereto which do not exceed the scope of the appended claims. Therefore, the scope of the invention is only to be limited by the following claims. In some embodiments, the figures presented in this patent application are drawn to scale, including the angles, ratios of dimensions, etc. In some embodiments, the figures are representative only and the claims are not limited by the dimensions of the figures. In some embodiments, descriptions of the inventions described herein using the phrase “comprising” includes embodiments that could be described as “consisting essentially of” or “consisting of”, and as such the written description requirement for claiming one or more embodiments of the present invention using the phrase “consisting essentially of” or “consisting of” is met.

The reference numbers recited in the below claims are solely for ease of examination of this patent application, and are exemplary, and are not intended in any way to limit the scope of the claims to the particular features having the corresponding reference numbers in the drawings.

What is claimed is:

1. A portable device (100) for detection of cerebrospinal fluid (CSF) in a sample, the device (100) comprising:
a. a substrate (110);
b. a sensor (120) disposed on the substrate (110), the sensor (120) comprising a sensing area;
wherein the sensor (120) further comprises one or more aptamers disposed on a surface of the sensor (120), wherein the one or more aptamers are configured to selectively bind to one or more molecules found in CSF; and
c. an electrochemical detection component (130) operatively coupled to the sensor (120);
wherein, when a sample containing an amount of the one or more molecules found in CSF is disposed on the sensing area of the sensor (120), the sensor (120) is configured to generate a detection signal to be transmitted to the electrochemical detection component (130).

2. The device (100) of claim 1, wherein the sensor (120) comprises a miniaturized reference electrode, a miniaturized working electrode, and a miniaturized counter electrode.

3. The device (100) of claim 2, wherein the miniaturized reference electrode comprises a wrinkled reference electrode, wherein the miniaturized working electrode comprises a wrinkled working electrode, wherein the miniaturized counter electrode comprises a wrinkled counter electrode.

4. The device (100) of claim 3, wherein the wrinkled reference electrode, the wrinkled working electrode, and the wrinkled counter electrode each comprise a thin shrunken layer of gold sputtered onto polystyrene plastic.

5. The device (100) of claim 2, wherein the miniaturized reference electrode, the miniaturized working electrode, and the miniaturized counter electrode comprise nanoparticle electrodes.

6. The device (100) of claim 1, wherein the one or more aptamers are modified by a redox agent that functionalizes a first end of each aptamer to be sensitive to the one or more molecules in CSF.

7. The device (100) of claim 6, wherein the redox agent comprises methylene blue, ferrocene, or a combination thereof.

8. The device (100) of claim 6, wherein a second end of each aptamer is functionalized to attach to the surface of the sensor (120).

9. The device (100) of claim 1, wherein the electrochemical detection component (130) is configured to connect to a computing device (140) and transmit the detection signal to the computing device (140).

10. The device (100) of claim 1, wherein the sensor (120) is configured to sense 1 ag/mL or more of the one or more molecules found in CSF.

11. The device of claim 1, wherein the one or more aptamers are any one or a combination of SEQ ID NO: 1-6.

12. The device of claim 1, wherein the electrochemical detection component comprises a potentiostat, a multimeter, or a combination thereof.

13. A method for detecting cerebrospinal fluid (CSF) in a sample, the method comprising:
a. providing a device (100) comprising:
i. a substrate (110);
ii. a sensor (120) disposed on the substrate (110), the sensor (120) comprising a sensing area;
wherein the sensor (120) comprises an aptamer solution comprising an aptamer sequence comprising a first end and a second end, wherein the first end is functionalized by a redox agent to be sensitive to one or more molecules found in CSF, and wherein the second end is functionalized to attach to a surface of the sensor (120); and
iii. an electrochemical detection component (130) operatively coupled to the sensor (120);
b. applying a sample to the sensing area of the sensor (120);
c. generating, by the sensor (120), a detection signal if the sample contains an amount of the one or more molecules found in CSF; and
d. transmitting the detection signal to the electrochemical detection component (130).

14. The method of claim 13, wherein the sensor (120) comprises a miniaturized reference electrode, a miniaturized working electrode, and a miniaturized counter electrode.

15. The method of claim 14, wherein the miniaturized reference electrode, the miniaturized working electrode, and the miniaturized counter electrode each comprise a thin shrunken layer of gold sputtered onto polystyrene plastic.

16. The method of claim 13, wherein the aptamer solution is modified by a redox agent.

17. The method of claim 13, wherein the sensor (120) is configured to sense 1 ag/mL or more of the one or more molecules found in CSF.

18. An aptamer comprising a first end and a second end, wherein the first end is functionalized by a redox agent to be sensitive to one or more molecules found in cerebrospinal fluid (CSF), and wherein the second end is functionalized to attach to a surface of an electrode, wherein the aptamer is according to any one of SEQ ID NO: 1-6.

19. The aptamer sequence of claim 18, wherein the redox agent comprises methylene blue, ferrocene, any chemical capable of causing the aptamer to change electron transfer in response to the one or more molecules found in CSF, or a combination thereof.

20. The aptamer sequence of claim 18, wherein the second end is functionalized by thiolization, biotinylation, or a combination thereof.
